(19) United States
(12) Patent Application Publication
(10) Pub. No.: US 2012/0321671 A1
(43) Pub. Date: Dec. 20, 2012

(54) Method for reducing heat loss in neonatal mammalian subjects by transplanting brown adipocytes or precursors thereof

(75) Inventors: Edward S. Boyden, Chestnut Hill, MA (US); Roderick A. Hyde, Redmond, WA (US); Muriel Y. Ishikawa, Livermore, CA (US); Edward K.Y. Jung, Bellevue, WA (US); Eric C. Leuthardt, St. Louis, MO (US); Stephen L. Malaska, Redmond, WA (US); Elizabeth A. Sweeney, Seattle, WA (US); Lowell L. Wood, JR., Bellevue, WA (US); Victoria Y.H. Wood, Livermore, CA (US)

(73) Assignee: Searete LLC, a limited liability corporation of the State of Delaware

(21) Appl. No.: 13/134,854

(22) Filed: Jun. 16, 2011

(51) Int. Cl. A61K 35/12; A61K 9/14; A61K 35/14; A61K 35/50; A61K 35/34; A61K 35/28

(52) U.S. Cl. 424/400; 424/93.7; 424/93.21

(57) ABSTRACT

A method for modulating heat loss in a neonatal mammalian subject is disclosed herein that includes providing exogenous brown adipocytes, or precursors thereof, to an internal site in the neonatal mammalian subject.

### SUMMARY

Methods are disclosed herein for modulating heat loss in a neonatal mammalian subject. The method includes providing exogenous brown adipocytes, or precursors thereof, to an internal site in the neonatal mammalian subject. In the method disclosed herein, providing brown adipocytes can include providing pre-adipocytes or providing precursor cells of exogenous brown adipocytes to an internal site in the neonatal mammalian subject. The method can further include providing factors that promote at least one of adipocyte proliferation, adipocyte differentiation, or adipose angiogenesis. The factors can include, but are not limited to, differentiation factors, growth factors, or angiogenic factors. The factors that support or enhance proliferation can be configured to induce differentiation of the pre-adipocytes or adipocyte precursors to brown adipocytes. The internal site for placement of brown adipocytes in the neonatal mammalian subject can include one or more of a subcutaneous site, scapular site, axillary site, thoracic site, abdominal site, or blood vessel site of the neonatal mammalian subject. In some aspects, the brown adipocytes, or precursors thereof, can be substantially purified. The neonatal mammalian subject can include, but is not limited to, a human, equine, bovine, ovine, swine, rodent, lagomorph, canine, or feline. The neonatal mammalian subject can include a neonatal mammalian subject born preterm.

In some aspects, modulating heat loss includes reducing heat loss in the neonatal mammalian subject by providing the exogenous brown adipocytes, or the precursors thereof, to the internal site in the neonatal mammalian subject. The precursors can include, but are not limited to, one or more of stem cells, totipotent stem cells, multipotent stem cells, pluripotent stem cells, oligopotent stem cells, embryonic stem cells, de-differentiated stem cells, trans-differentiated stem cells, mesenchymal stem cells, adipose-derived stem cells, adipocyte progenitor cells, pre-adipocytes, myoblasts, muscle-derived stem cells, or bone marrow-derived stem cells. The exogenous brown adipocytes can include mature brown adipocytes. The internal site can include, but is not limited to, one or more of a subcutaneous site, scapular site, axillary site, thoracic site, abdominal site, or blood vessel site of the neonatal mammalian subject. The brown adipocytes or the precursors thereof can be derived from one or more of cell donors, tissue donors, tissue culture stock, cell lines, or genetically manipulated cells. The genetically manipulated cells can include an exogenous DNA sequence encoding one or more of a mammalian UCP polypeptide or a PRDM16 polypeptide.

The one or more cell donors or tissue donors can include a genetically related donor of the neonatal mammalian subject. The genetically related donor can include, but is not limited to, a mother, father, sibling, grandparent, aunt, or uncle of the neonatal mammalian subject. The exogenous brown adipocytes or the precursors thereof can be derived from one or more of an autologous tissue, allogeneic tissue, or xenogeneic tissue. The exogenous brown adipocytes or the precursors thereof can be derived from a neonatal-associated tissue. The exogenous brown adipocytes or the precursors thereof can be derived from one or more of adipocytes, pre-adipocytes, stem cells, cord blood cells, placental cells, myoblasts, or bone marrow cells. The method can further include expanding, maturing, or differentiating the exogenous brown adipocytes or the precursors thereof in vitro. The method can further include providing one of more of differentiation factors or growth factors in vitro to the exogenous brown adipocytes or the precursors thereof. The brown adipocytes, or the precursors thereof further can include one or more detectable markers incorporated with the brown adipocytes or the precursors thereof. In some aspects, providing the brown adipocytes or the precursors thereof to the internal site can include injecting the brown adipocytes or the precursors thereof. The method can further include injecting the brown adipocytes or the precursors thereof in a pharmaceutically acceptable carrier. In some aspects, providing the brown adipocytes or the precursors thereof to the internal site can include implanting the brown adipocytes or the precursors thereof. The method can further include providing the brown adipocytes or the precursors thereof in a pharmaceutically acceptable carrier. The method can further include providing the brown adipocytes or the precursors thereof in one or more biocompatible carriers.

The method can further include encapsulating the brown adipocytes or the precursors thereof. The method can further include providing the brown adipocytes or the precursors thereof in an immunoisolating material. The biocompatible carrier can include, but is not limited to, at least one of a membrane, natural matrix, synthetic matrix, polymer, scaffold, hydrogel, natural sponge, synthetic sponge, microbead, microcapsule, microsphere, microparticle, or an encapsulating material. The method can further include providing one or more medicaments for modulating heat loss from the brown adipocytes. The one or more medicaments can include, but is not limited to, one or more of a neurotransmitter, a neurotrophic agent, a neuropeptide, an adipokine, or an uncoupling protein. The one or more medicaments can include, but is not limited to, one or more of a β3-adrenergic receptor agonist, NPY antagonist, leptin, UCP activating agent, thyroxine, serotonin reuptake inhibitor, MCH antagonist, GLP-1 agonist, 5-HT2C agonist, 5-HT2A agonist, galanin antagonist, CRF agonist, urocortin agonist, melanocortin agonist or enterostatin agonist.

A method is disclosed herein that includes: providing exogenous brown adipocytes, or precursors thereof, to an internal site in a neonatal mammalian subject. In some aspects, providing the brown adipocytes can include providing pre-adipocytes to the neonatal mammalian subject. The brown adipocytes, or the precursors thereof, can be substantially purified. The method can further include providing factors to the subject that promote at least one of adipocyte proliferation, adipocyte differentiation, or adipose angiogenesis. The factors can include, but are not limited to, differentiation factors, growth factors, or angiogenic factors. The neonatal mammalian subject can include, but is not limited to, a human, equine, bovine, ovine, swine, rodent, lagomorph, canine, or feline. The neonatal mammalian subject can include a neonatal mammalian subject born preterm.

In some aspects, modulating heat loss includes reducing heat loss in the neonatal mammalian subject by providing the exogenous brown adipocytes, or the precursors thereof, to the internal site in the neonatal mammalian subject. The precursors can include, but are not limited to, one or more of stem cells, totipotent stem cells, multipotent stem cells, pluripotent stem cells, oligopotent stem cells, embryonic stem cells, de-differentiated stem cells, trans-differentiated stem cells, mesenchymal stem cells, adipose-derived stem cells, adipocyte progenitor cells, pre-adipocytes, myoblasts, muscle-derived stem cells, or bone marrow-derived stem cells. The exogenous brown adipocytes can include mature brown adipocytes. The internal site can include, but is not limited to, one or more of a subcutaneous site, scapular site, axillary site, thoracic site, abdominal site, or blood vessel site of the neonatal mammalian subject. The brown adipocytes or the precursors thereof can be derived from one or more of cell donors, tissue donors, tissue culture stock, cell lines, or genetically manipulated cells. The genetically manipulated cells can include an exogenous DNA sequence encoding one or more of a mammalian UCP polypeptide or a PRDM16 polypeptide.

The one or more cell donors or tissue donors can include a genetically related donor of the neonatal mammalian subject. The genetically related donor can include, but is not limited to, a mother, father, sibling, grandparent, aunt, or uncle of the neonatal mammalian subject. The exogenous brown adipocytes or the precursors thereof can be derived from one or more of an autologous tissue, allogeneic tissue, or xenogeneic tissue. The exogenous brown adipocytes or the precursors thereof can be derived from a neonatal-associated tissue. The exogenous brown adipocytes or the precursors thereof can be derived from one or more of adipocytes, pre-adipocytes, stem cells, cord blood cells, placental cells, myoblasts, or bone marrow cells. The method can further include expanding, maturing, or differentiating the exogenous brown adipocytes or the precursors thereof in vitro. The method can further include providing one of more of differentiation factors or growth factors in vitro to the exogenous brown adipocytes or the precursors thereof. The brown adipocytes, or the precursors thereof further can include one or more detectable markers incorporated with the brown adipocytes or the precursors thereof. In some aspects, providing the brown adipocytes or the precursors thereof to the internal site can include injecting the brown adipocytes or the precursors thereof. The method can further include injecting the brown adipocytes or the precursors thereof in a pharmaceutically acceptable carrier. In some aspects, providing the brown adipocytes or the precursors thereof to the internal site can include implanting the brown adipocytes or the precursors thereof. The method can further include providing the brown adipocytes or the precursors thereof in a pharmaceutically acceptable carrier. The method can further include providing the brown adipocytes or the precursors thereof in one or more biocompatible carriers.

The method can further include encapsulating the brown adipocytes or the precursors thereof. The method can further include providing the brown adipocytes or the precursors thereof in an immunoisolating material. The biocompatible carrier can include, but is not limited to, at least one of a membrane, natural matrix, synthetic matrix, polymer, scaffold, hydrogel, natural sponge, synthetic sponge, microbead, microcapsule, microsphere, microparticle, or an encapsulating material. The method can further include providing one or more medicaments for modulating heat loss from the brown adipocytes. The one or more medicaments can include, but is not limited to, one or more of a neurotransmitter, a neurotrophic agent, a neuropeptide, an adipokine, or an uncoupling protein. The one or more medicaments can include, but is not limited to, one or more of a β3-adrenergic receptor agonist, NPY antagonist, leptin, UCP activating agent, thyroxine, serotonin reuptake inhibitor, MCH antagonist, GLP-1 agonist, 5-HT2C agonist, 5-HT2A agonist, galanin antagonist, CRF agonist, urocortin agonist, melanocortin agonist or enterostatin agonist.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

FIGS. 1A and 1B are schematics of a diagrammatic view of an aspect of an embodiment of a method for modulating heat loss in a neonatal mammalian subject.

FIGS. 2A and 2B are schematics of a diagrammatic view of an aspect of an embodiment of a method for modulating heat loss in a neonatal mammalian subject.

FIG. 3 is a schematic of a diagrammatic view of an aspect of an embodiment of a method for modulating heat loss in a neonatal mammalian subject.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

This document uses formal outline headings for clarity of presentation. However, it is to be understood that the outline headings are for presentation purposes, and that different types of subject matter may be discussed throughout the application (e.g., method(s) may be described under composition heading(s) and/or kit headings, and/or descriptions of single topics may span two or more topic headings). Hence, the use of the formal outline headings is not intended to be in any way limiting.

Methods are disclosed herein for modulating heat loss in a neonatal mammalian subject. The method includes providing exogenous brown adipocytes, or precursors thereof, to an internal site, e.g., in vivo, in the neonatal mammalian subject. In the method disclosed herein, providing brown adipocytes can include providing pre-adipocytes or providing precursor cells of exogenous brown adipocytes to an internal site in the neonatal mammalian subject. The method can further include providing factors that promote at least one of adipocyte proliferation, adipocyte differentiation, or adipose tissue angiogenesis. The factors can include, but are not limited to, differentiation factors, growth factors, or angiogenic factors. Promoting at least one of adipocyte proliferation, adipocyte differentiation, or adipose tissue angiogenesis can include, but is not limited to, inducing, supporting, or enhancing at least one of adipocyte proliferation, adipocyte differentiation, or adipose angiogenesis. The factors that promote proliferation can be formulated to induce support, or enhance differentiation of the pre-adipocytes or adipocyte precursors to brown adipocytes. The internal site for placement of brown adipocytes in the neonatal mammalian subject can include one or more of a subcutaneous site, scapular site, axillary site, thoracic site, abdominal site, or blood vessel site of the neonatal mammalian subject. The internal site for placement of brown adipocytes in the neonatal mammalian subject can further include regions of the neck, regions of the back including back regions along the spine and above the buttock, and regions of the scalp of the neonatal mammalian subject. In some aspects, the brown adipocytes or precursors thereof can be substantially purified.

Methods are disclosed herein for modulating heat loss in a neonatal mammalian subject. Modulating heat loss in the neonatal mammalian subject can include, but is not limited to, decreasing the rate of heat loss or the amount of heat loss in the neonatal mammalian subject, or controlling a decrease or an increase in the rate of heat loss or the amount of heat loss in the neonatal mammalian subject. The method includes providing exogenous brown adipocytes, or precursors thereof, to an internal site, e.g., in vivo, in the neonatal mammalian subject. In the method disclosed herein, providing exogenous brown adipocytes can include providing exogenous pre-adipocytes or providing exogenous precursor cells or exogenous progenitor cells of brown adipocytes to an internal site in the neonatal mammalian subject. The precursors can include, but are not limited to, stem cells, totipotent stem cells, multipotent stem cells, pluripotent stem cells, oligopotent stem cells, embryonic stem cells, de-differentiated stem cells, trans-differentiated stem cells, mesenchymal stem cells, adipose-derived stem cells, adipocyte progenitor cells, pre-adipocytes, myoblasts, muscle-derived stem cells, or bone marrow-derived stem cells. Precursors of brown adipocytes include, but are not limited to, stem cells or progenitor cells that are in any stage of development or differentiation. Stem cells include, but are not limited to, cells that can proliferate and differentiate through developmental stages into mature cell types. Progenitor cells include cell descendants of stem cells that are committed to a developmental lineage. Pre-adipocyte includes a specific progenitor cell of a brown adipocyte or a white adipocyte.

The method can further include providing factors that promote proliferation of brown adipocytes or precursors thereof. The method can further include providing factors that promote or induce adipocyte differentiation. The method can further include providing factors that promote angiogenesis of adipose tissue. Factors provided can include, but are not limited to, differentiation factors, growth factors, or angiogenic factors. The differentiation factors, growth factors, or angiogenic factors can be provided in vitro to cells in culture prior to providing the cells to the neonatal mammalian subject. Alternatively, the differentiation factors, growth factors, or angiogenic factors can be provided in vivo with the exogenous cells at an internal site in the neonatal mammalian subject. Various growth or angiogenic factors, e.g., basic fibroblast growth factor (bFGF), acidic fibroblast growth factor, and vascular endothelial growth factor (VEGF) can also be administered as an adjunct to improve vascularization of the tissue comprising the brown adipocyte transplant. The differentiation factors, growth factors, or angiogenic factors can be provided to the same site as the exogenous cells in the neonatal mammalian subject, prior to, at the same time, or after a time interval from providing the exogenous cells. The differentiation factors, growth factors, or angiogenic factors can be provided to a different site in the neonatal mammalian subject than the site to which the exogenous cells have been provided. The factors can be provided prior to, at the same time, or after a time interval from providing the exogenous cells. The factors can be provided to the neonatal mammalian subject systemically prior to, at the same time, or after a time interval from providing the exogenous cells. The internal site for placement of brown adipocytes in the neonatal mammalian subject can include one or more in vivo sites in the neonatal mammalian subject including one or more of a subcutaneous site, scapular site, axillary site, thoracic site, abdominal site, or blood vessel site of the neonatal mammalian subject. In some aspects, the brown adipocytes, or precursors thereof, can be substantially purified.

The neonatal mammalian subject can include, but is not limited to, a subject that is human, equine, bovine, ovine, swine, rodent, lagomorph, canine, or feline. The non-human neonatal mammalian subjects can be used, for example, in areas of husbandry, companion animals, purebred show animals, or laboratory animals.

A method is described herein for modulating heat loss in a neonatal mammalian subject by providing exogenous thermogenic brown adipocytes to an internal site in the neonatal subject. In general, mammalian neonates sustain life within a narrow range of core body temperature; for humans this is generally from about 36.5° C. to about 37.5° C. The mammalian body uses a number of mechanisms to balance heat loss and heat production in order to maintain the core temperature within this range despite a wide range of environmental temperatures. Balancing heat loss and gain is known as thermoregulation. The range of ambient temperature that maintains a mammalian neonate's core body temperature using minimum oxygen consumption at a minimum metabolic rate is called the thermoneutral range. See, e.g., Asakura, J. Nippon Med. Sch. 71: 360-370, 2004; which is incorporated herein by reference.

At birth, a human neonate's thermoregulatory mechanisms are not fully developed, but the thermoregulatory mechanisms become progressively more efficient during early development. Neonates exhibit limited response to temperature changes in the first 24 hours of life and are particularly susceptible to chilling during this time. By 24-48 hours of age, healthy term neonates are able to increase their heat production up to 2.5 times in response to cold; however, they remain susceptible to environmental temperature changes. Until the mechanisms controlling thermoregulation (and heat loss in particular) stabilize, the thermoneutral range of infants is at a higher environmental temperature than older children and adults. The method, as described herein, for modulating heat loss in a neonatal mammalian subject by providing exogenous thermogenic brown adipocytes to an internal site in the neonatal subject can be used to enhance thermoregulatory mechanisms in the first weeks of life of a human neonate.

Heat is easily lost at birth, when autonomic thermoregulation is at its least efficient. Hypothermia (e.g., a core temperature of less than 36.5° C. in humans) can occur rapidly unless active steps are taken to prevent heat loss, and perinatal hypothermia is a contributing factor to adverse outcomes. The World Health Organization classifies a newborn human core body temperature of 36.0 to 36.4 C° as mild hypothermia, 32 to 35.9 C° as moderate hypothermia, and lower than 32 C° as severe hypothermia. The signs, symptoms, and complications of hypothermia in a neonate can include, but are not limited to, body cool to touch, mottling or pallor, central cyanosis, acrocyanosis, poor feeding, abdominal distension, hypotonia, hypoglycemia, increased gastric residuals, bradycardia, tachypnea, restlessness, shallow or irregular respiration, apnea, and lethargy. See, e.g., Bhatt, et al., J. Perinatology 27: S45-S47, 2007, which is incorporated herein by reference.

A number of features contribute to ease of heat loss in neonates, including a large surface area-to-mass ratio (in humans approximately three times greater than adults); less subcutaneous fat; thin epidermis; blood vessels closer to the skin allowing changes in ambient temperature to more readily affect the circulating blood, thereby influencing the temperature-regulating center in the hypothalamus; and a decreased ability to shiver. In contrast, several features contribute to heat production in full-term neonates including a decreased ability to sweat, a flexed posture which conserves heat by reducing exposed surface area, and the ability to generate heat by non-shivering thermogenesis. In preterm neonates, these features are not fully developed, making it even more difficult for the preterm neonates to maintain a normal body temperature.

Non-shivering thermogenesis is facilitated by increased metabolic activity in brown adipose tissue (BAT) resulting in the generation of heat. Brown adipose tissue is so-named because it has a darker hue than white adipose tissue due to a more extensive blood supply and greater numbers of mitochondria. In response to cooling, signals are sent from the central nervous system to the brown adipose tissue to induce lipolysis, the enzymatic breakdown of trigylcerides to fatty acids. Lipolysis in most cell types results in the generation of ATP. However, in brown adipocytes, lipolysis is uncoupled from the production of ATP, releasing the energy as heat. Uncoupling protein 1 (UCP-1) plays a central role in facilitating the uncoupling process and the release of heat. Oxygen is required for lipolysis, and lipolysis in brown adipose tissue uses up to three times as much oxygen as other tissue. See, e.g., Nedergaard & Cannon, Cell Metab. 11 (4): 268-272, 2010; Cannon & Nedergaard, “Brown adipose tissue: Function and physiological significance,” Physiol. Rev. 84:277-359, 2004; Morrison & Nakamura, Frontiers in Bioscience 16: 74-104, 2011; Bartness, et al., International Journal of Obesity 34: S36-S42, 2010; Lee, et al., Am J Physiol Endocrinol Metab 299: E601-E606, 2010; Saely, et al., “Brown versus White Adipose Tissue: A Mini-Review,” Gerontology, Karger AG, Basel, Dec. 7, 2010; Bartell et al., Nat Med (2011) January 23, doi: 10.1038/nm.2297; Ma et al., PLoS ONE 6 (1): e16391. doi:10.1371/journal.pone.0016391. 2011; Richard and Picard, Frontiers in Bioscience 16: 1233-1260, 2011; which are incorporated herein by reference.

BAT is thought to constitute 2-7% of total body weight in human neonatal subjects. BAT is deposited in the fetus beginning at 26 weeks of gestation, steadily increasing in amount until two to five week after birth. At term, BAT is deposited around the nape of the neck and mid-scapular area, under the clavicles and in the axillae, around the kidneys, adrenal glands and large vessels in the neck and in the mediastinum. See, e.g., Richard and Picard, Frontiers in Bioscience 16: 1233-1260, 2011; Heaton, J. Anat., 112: 35-39, 1972, which are incorporated herein by reference. Maternal prostaglandins and adenosine prevent non-shivering thermogenesis in utero; BAT is activated after birth.

Preterm neonates and small-for-gestational-age neonates have less brown adipose tissue and a greater surface area to mass ratio than full-term neonates. Furthermore, the lower the gestational age, the less brown adipose tissue is available for thermoregulation. In addition, the muscles of preterm neonates are less developed, resulting in less flexing of the body and limbs. Consequently, these infants are extremely vulnerable to hypothermia. In general, maintaining body temperature is an integral part of treatment in the preterm neonate and is extremely challenging. Low temperature on admission to the neonatal intensive care is an independent risk factor for mortality in extremely preterm neonates. See, e.g, Costeloe, et al., Pediatrics 106: 659-671 2000; Lyon, et al., Arch. Dis. Child. 76: F47-F50, 1997, which are incorporated herein by reference. The method, as described herein, for modulating heat loss in a neonatal mammalian subject by providing exogenous thermogenic brown adipocytes to an internal site in the neonatal subject can be used as part of a treatment for hypothermia in preterm neonates.

Low birth weight in human neonates and in nonhuman neonatal mammals, e.g., domesticated animals, is a condition that indicates less insulation for the neonatal mammal and also indicates a limited supply of tissue substrates available as a source for energy conversion. The method, as described herein, for modulating heat loss in a low birth weight neonatal mammalian subject by providing exogenous thermogenic brown adipocytes to an internal site in the neonatal mammalian subject can be used alone or in combination with administration of nutritional supplements such as glucose to the neonatal mammalian subject. The single treatment or the combination treatment can be used to treat low birth weight in preterm neonates or in full term neonates.

The method, as described herein, for modulating heat loss in a low birth weight neonatal mammalian subject by providing exogenous thermogenic brown adipocytes to an internal site in the neonatal mammalian subject can be used to prevent starvation in nonhuman neonatal mammalian subjects, e.g., when the neonate has not being accepted for nursing by its mother. The method, as described herein, can be used to treat conditions in human or nonhuman mammalian neonatal subjects including, but not limited to, dystocia (difficult birth); in utero exposure to drugs or alcohol, e.g., fetal alcohol syndrome, resulting in small-for-gestational-age birth and/or hypothermia; or neonatal recurrent prolonged hypothermia associated with maternal mirtazapine treatment during pregnancy. See, e.g., Can J Clin Pharmacol 15 (2): e188-e190, 2008, which is incorporated herein by reference. Fetal alcohol syndrome can have associated hypothermia and hypoglycemia.

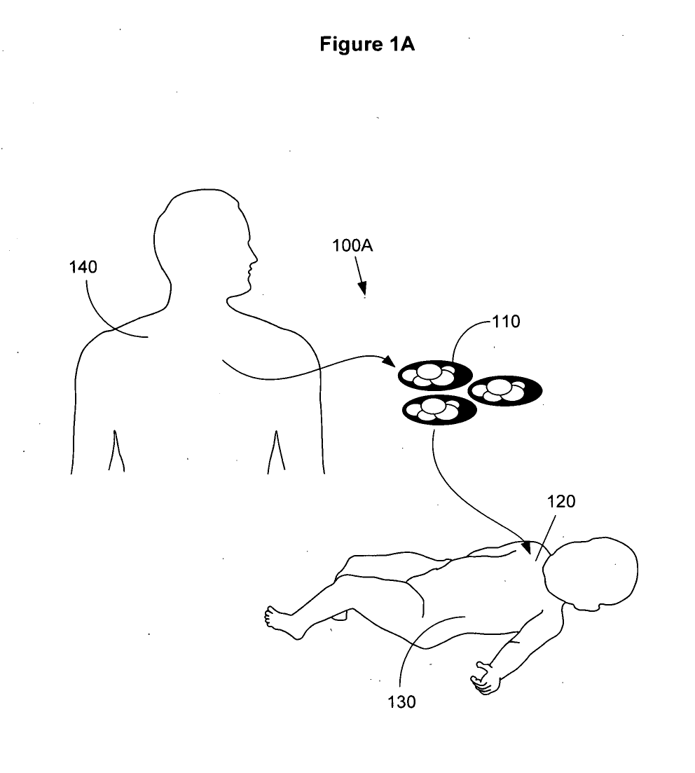

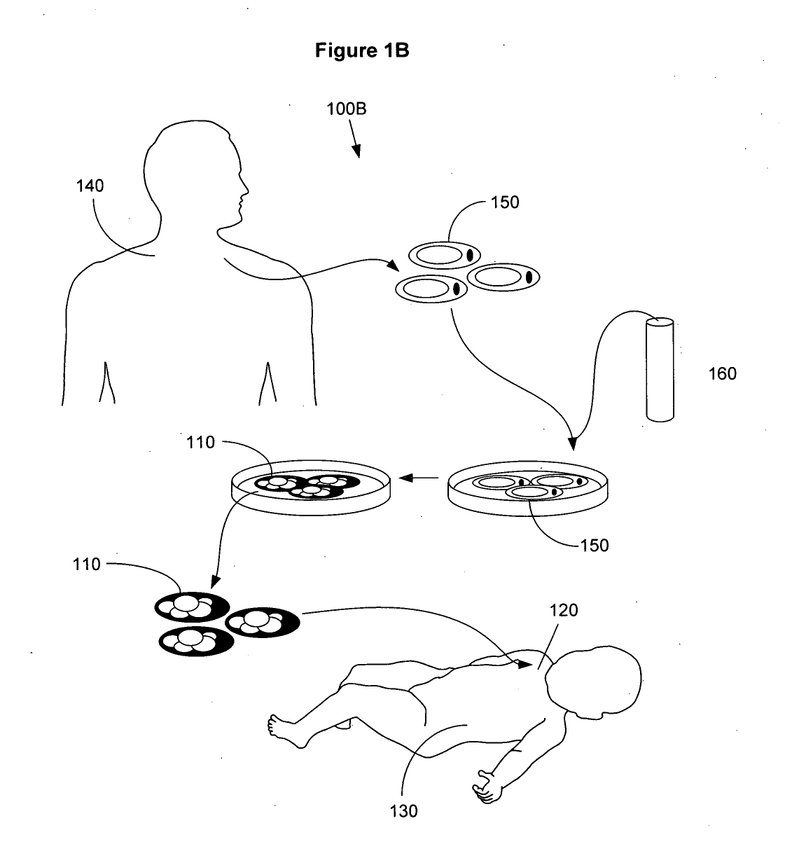

With reference to the figures, and with reference now to FIGS. 1, 2, and 3, depicted is an aspect of a method that can serve as an illustrative embodiment of and/or for subject matter technologies, for example a method for modulating heat loss in a neonatal mammalian subject that includes providing brown adipocytes, or precursors thereof, to an internal site in the neonatal mammalian subject. The specific methods disclosed herein are intended as merely illustrative of their more general counterparts.

Referring to FIG. 1A, depicted is a partial diagrammatic view of a method 100A for modulating heat loss in a neonatal mammalian subject that includes providing exogenous brown adipocytes 110 to an internal site 120 in the neonatal mammalian subject 130. Brown adipocytes 110, including adipocyte precursors and mature brown adipocytes, harvested from a supraclavicular region of an allogeneic donor 140, e.g., a genetically related donor such as the mother of the neonatal human subject, can be transplanted to an internal site 120 in the neonatal subject 130. In some aspects, the brown adipocytes 110, or the precursor cells or the progenitor cells thereof, from the allogeneic donor 140 can be substantially purified. The internal site 120 for injection of the brown adipocytes into the neonatal subject can be an interscapular region of the neonatal subject 130. The interscapular region for injection of the brown adipocytes into the neonatal subject may already include some brown adipose tissue.

Referring to FIG. 1B, depicted is a partial diagrammatic view of a method 100B for modulating heat loss in a neonatal mammalian subject 130 that includes providing brown adipocytes 110, or precursors or progenitors thereof 150, to an internal site 120 in the neonatal mammalian subject 130. Adipose tissue including pre-adipocytes 150 can be harvested from a subcutaneous region of an allogeneic donor 140, e.g., a genetically related donor such as the mother of the neonatal human subject. Cells from the adipose tissue including pre-adipocytes or multi-potent adipose-derived stem cells 150 can be cultured in vitro in the presence of growth factors to expand the cell number, and/or, e.g. subsequently, in the presence of differentiation factors 160 to induce differentiation into brown adipocytes 110. In some aspects, the brown adipocytes 110, or the precursor cells or the progenitor cells thereof, from the in vitro tissue culture can be substantially purified. The in vitro cultured brown adipocytes 110 can be transplanted to an internal site 120 in the neonatal subject 130. The internal site 120 of transplantation of the brown adipocytes into the neonatal subject can be an interscapular region of the neonatal subject 130.

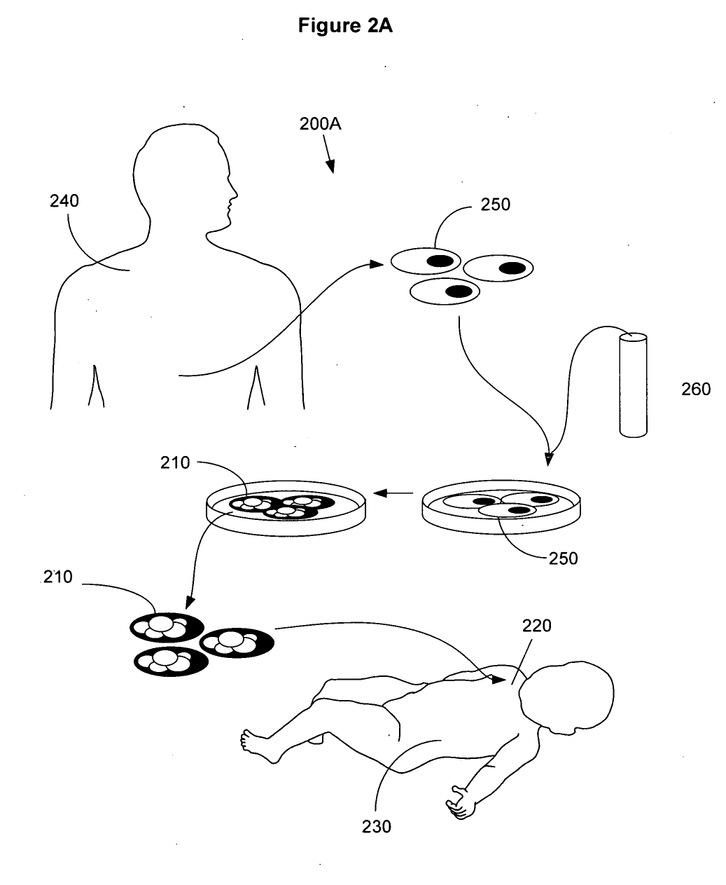

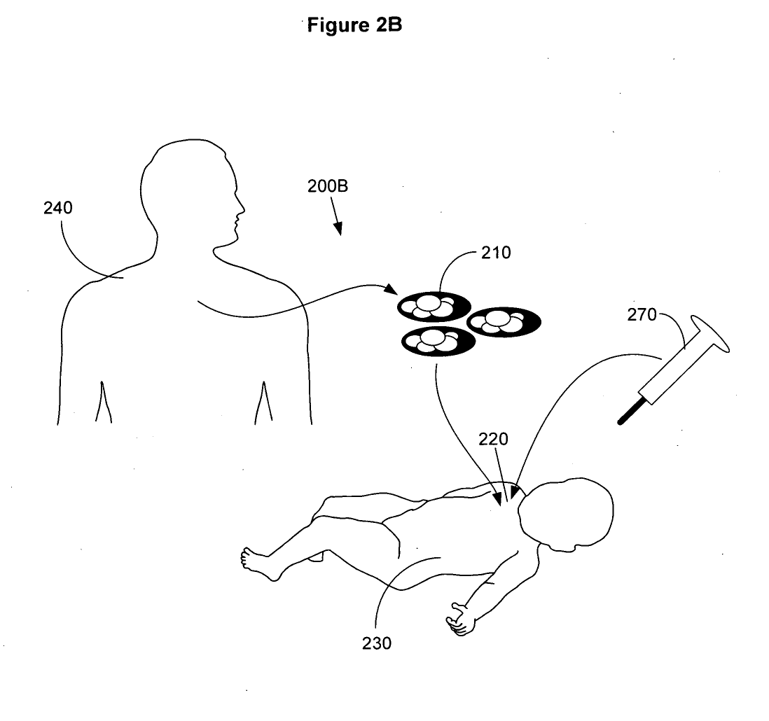

Referring to FIG. 2A, depicted is a partial diagrammatic view of a method 200A for modulating heat loss in a neonatal mammalian subject 230 that includes providing brown adipocytes 210, or precursors or progenitors thereof 250, to an internal site 220 in the neonatal mammalian subject 230. Umbilical cord blood including umbilical cord stem cells 250 can be harvested from an umbilical cord, for example the umbilical cord of a neonatal human subject collected at birth. Alternatively, umbilical cord blood including umbilical cord stem cells 250 can be harvested from an umbilical cord of a donor 240, e.g., a different neonatal mammal such as monozygotic or dizygotic twin of the neonatal human subject, or an unrelated donor. For example, umbilical cord stem cells 250 can be harvested from an umbilical cord of a donor 240, stored, and revived for use in the neonatal mammalian subject 230. The umbilical cord blood including umbilical cord stem cells 250 can be cultured in vitro in the presence of factors 260, for example growth factors to expand the cell number and/or, e.g. subsequently, differentiation factors to induce differentiation into brown adipocytes 210. In some aspects, the brown adipocytes 210, or the precursors thereof, can be substantially purified from cells of umbilical cord blood prior to or subsequent to culture. The in vitro cultured brown adipocytes 210 can be transplanted to an internal site 220 in the neonatal subject 230. The internal site 220 of transplantation of the brown adipocytes into the neonatal subject can be an interscapular region of the neonatal subject 230.

Referring to FIG. 2B, depicted is a partial diagrammatic view of a method 200B for modulating heat loss in a neonatal mammalian subject 230 that includes providing brown adipocytes 210, or precursors or progenitors thereof, to an internal site 220 in the neonatal mammalian subject 230. Brown adipocytes 210 harvested from a supraclavicular region of an allogeneic donor 240, e.g. a genetically related donor such as the mother of the neonatal human subject, can be transplanted to an internal site 220 in the neonatal subject 230. In some aspects, the brown adipocytes 210, or the precursors or progenitors thereof, from the allogeneic donor 240 can be substantially purified. The internal site 220 for transplantation of the brown adipocytes into the neonatal subject can be an interscapular region of the neonatal subject 230. A medicament 270 formulated to modulate, e.g. induce, enhance, or control, thermoregulation can be administered to the neonatal subject 230 in combination with the transplanted brown adipose tissue 210.

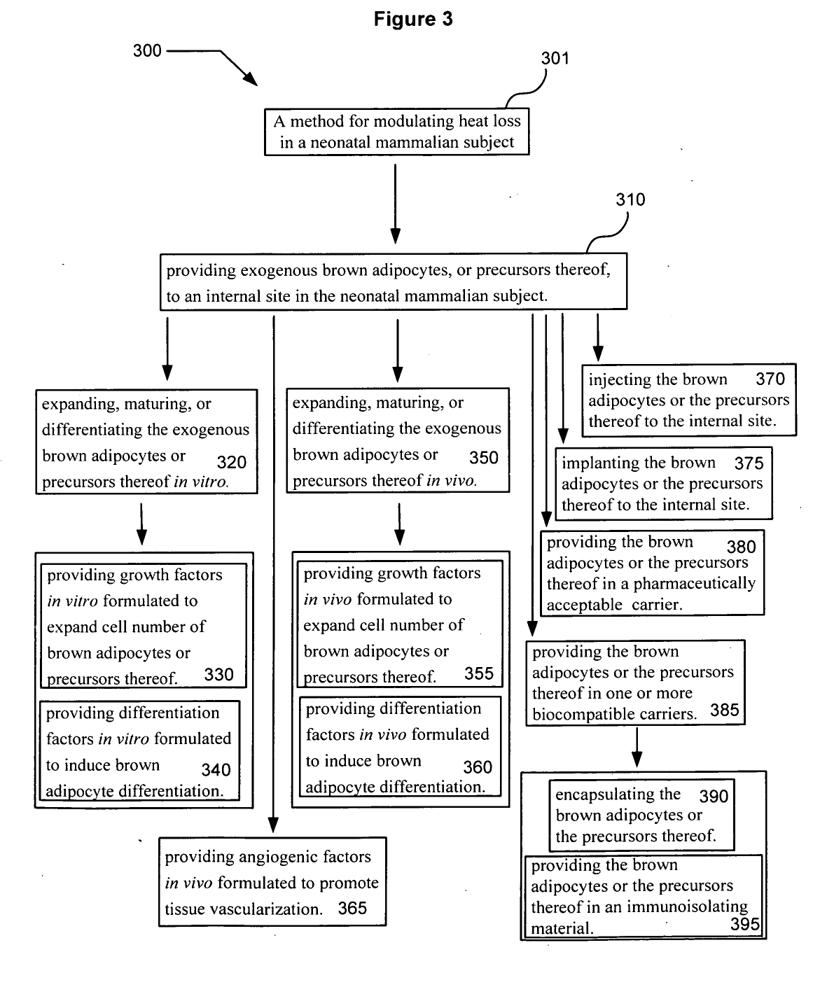

Referring to FIG. 3, depicted is a partial diagrammatic view of a method 300 for modulating heat loss in a neonatal mammalian subject 301 that includes providing exogenous brown adipocytes 310, or precursors thereof, to an internal site in the neonatal mammalian subject. The method can include providing brown adipocytes 310 or precursors thereof and providing in vitro or in vivo, e.g., prior to, simultaneously, or subsequently, exogenous factors 320, 330, 340 with the brown adipocytes in the neonatal mammalian subject. The method can include expanding, maturing, or differentiating 320 the exogenous brown adipocytes or precursors thereof in vitro. The method can include providing growth factors in vitro 330 formulated to expand cell number of brown adipocytes or precursors thereof or providing differentiation factors in vitro 340 formulated to induce brown adipocyte differentiation. The method can include expanding, maturing, or differentiating 350 the exogenous brown adipocytes or precursors thereof in vivo. The method can include providing growth factors in vivo 355 formulated to expand cell number of brown adipocytes or precursors thereof or providing differentiation factors in vivo 360 formulated to induce brown adipocyte differentiation. The method can further include providing to the neonatal mammalian subject angiogenic factors in vivo 365 formulated to promote tissue vascularization.

The method for modulating heat loss can include reducing heat loss in the neonatal mammalian subject by providing the exogenous brown adipocytes, or the precursors thereof, to the internal site in the neonatal mammalian subject. The method can include injecting 370 the brown adipocytes or the precursors thereof to the internal site. The method can include implanting 375 the brown adipocytes or the precursors thereof to the internal site. The method can include providing 380 the brown adipocytes or the precursors thereof in a pharmaceutically acceptable carrier. The method can include providing 385 the brown adipocytes or the precursors thereof in one or more biocompatible carriers which can include encapsulating 390 the brown adipocytes or the precursors thereof or providing 395 the brown adipocytes or the precursors thereof in an immunoisolating material.

### Source of Brown Adipocytes For Use In Transplantation

A method for modulating heat loss in a neonatal mammalian subject is described herein that includes providing exogenous brown adipocytes, or precursors thereof, to an internal site in the neonatal mammalian subject. Exogenous brown adipocytes for internal transplantation can be derived from a variety of sources including, but not limited to, brown adipose tissue extracted from a donor or brown adipocytes derived from precursors, e.g., pre-adipocytes, progenitor cells, or stem cells. The method can include providing differentiation factors or growth factors, in vivo or in vitro, or angiogenic factors in vivo, to induce brown adipocyte expansion and/or differentiation of progenitor cells or stem cells and adipocytes or adipose tissue derived therefrom. The progenitor or stem cells can include, but are not limited to, stem cells, totipotent stem cells, multipotent stem cells, pluripotent stem cells, oligopotent stem cells, embryonic stem cells, de-differentiated stem cells, trans-differentiated stem cells, mesenchymal stem cells, adipose-derived stem cells, adipocyte progenitor cells, pre-adipocytes, myoblasts, muscle-derived stem cells, or bone marrow-derived stem cells. See, e.g., Elabd, et al., Stem Cells 27: 2753-2760, 2009; Gomillion & Burg, Biomaterials 27: 6052-6063, 2006, which are incorporated herein by reference. Pre-adipocytes are also available from commercial sources (e.g., from Zen-Bio, Research Triangle Park, N.C.; Genlantis, San Diego, Calif.).

Exogenous brown adipocytes for transplantation into a neonatal subject can be derived from brown adipose tissue isolated from a donor. In some aspects, the brown adipocytes, e.g., mature brown adipocytes or adipose-derived stem cells or progenitor cells, can be substantially purified. The brown adipose tissue can be isolated from autologous tissue or from an allogeneic or xenogeneic tissue donor. The donor can be genetically related, either closely, e.g., a biological mother, father, and/or sibling, or more distantly, e.g., a grandparent, aunt, and/or uncle, or can be an unrelated individual. In some instances, the brown adipose tissue can be derived from a xenogeneic tissue donor.

Brown adipose tissue in small mammals is located primarily in the interscapular region and the axillae (i.e., underarm) and to a lesser degree near the thymus and in the dorsal midline region of the thorax and abdomen. A similar distribution of brown adipose tissue is observed in full-term human neonates. In adult mammals, e.g., adult humans, depots of functional brown adipose tissue are located in a region extending from the anterior neck to the thorax, and primarily in the supraclavicular region, as well as the cervical and mediastinal regions. Additional depots are found associated with blood vessels and some internal organs, e.g, kidneys. See, e.g., Virtanen, et al., N. Engl. J. Med., 360: 1518-1525, 2009; Cypess, et al., N. Engl. J. Med., 360: 1509-1517, 2009; and van Marken Lichtenberg et al., N. Engl. J. Med., 360: 1500-1508, 2009, which are incorporated herein by reference. Brown adipose tissue for transplantation can be resected from a donor by biopsy, surgery, or liposuction. See, e.g., U.S. Patent Application No. 2010/0015104, which is incorporated herein by reference.

Stem cell refers to a cell having the capacity to self-renew and to differentiate into mature, specialized cells. A totipotent stem cell can differentiate into any type of body cell in addition to all of the extraembryonic cell types. A pluripotent stem cell can differentiate into any type of body cell. A multipotent stem cell can differentiate into multiple cell types of a particular tissue, organ, or physiological system. A progenitor cell is a stem cell descendent that can differentiate to give rise to a cell in a distinct lineage. In particular, brown adipocyte progenitor cell, e.g., a pre-adipocyte or an early progenitor cell, refers to a cell with the potential to differentiate into brown adipocytes.

Brown adipocytes for transplantation into a neonatal subject can be derived from differentiation of pre-adipocytes. Pre-adipocytes are progenitor cells committed to the adipocyte lineage. Exposure of pre-adipocytes to differentiation factors results in morphological and biochemical changes including cell rounding and accumulation of triacylglycerol and lipid vacuoles. Pre-adipocytes can be isolated from adipose tissue removed from a subject or donor by biopsy, surgery or aspiration. In an aspect, the pre-adipocytes can be obtained from the subcutaneous fat of the neonate subject for autologous cell transplantation. Alternatively, the pre-adipocytes can be obtained from an appropriate donor for allogeneic cell transplantation. The pre-adipocytes can be obtained from a genetically related donor, e.g., a biological mother, father, or sibling, and prepared for transplantation either prior to or shortly after the birth of the neonatal subject. The pre-adipocytes can be isolated from adipose tissue after disruption and digestion, e.g., with one or more enzymes. Immunoselection and/or depletion can be used to further select for CD34⁺/CD31⁻ pre-adipocytes. See, e.g., Elabd, et al., Stem Cells 27: 2753-2760, 2009; Gomillion & Burg, Biomaterials 27: 6052-6063, 2006, which are incorporated herein by reference. Pre-adipocytes are also available from commercial sources (e.g., from Zen-Bio, Research Triangle Park, N.C.; Genlantis, San Diego, Calif.).

Pre-adipocytes isolated from white adipose tissue or brown adipose tissue can be cultured in differentiation medium containing one or more of insulin, isobutyl methylxanthine (IBMX), dexamethasone, and transferrin. Pre-adipocytes can be differentiated into brown adipocytes in the presence of a PPARγ (peroxisome proliferator-activated receptor γ) selective agonist, e.g., rosiglitazone. See, e.g., Elabd, et al., Stem Cells 27: 2753-2760, 2009, which is incorporated herein by reference. Pre-adipocytes derived from subcutaneous white adipose tissue or brown adipose tissue can in addition or alternatively be cultured in the presence of one or more bone morphogenetic protein (BMP), such as BMP2, BMP4, BMP6 or BMP7, which can promote differentiation into brown adipocytes; in particular BMP7 can be added to the culture medium to promote differentiation into brown adipocytes. (See, e.g., Tseng et al., Nature 454 (7207): 1000-1004, 2008, which is hereby incorporated by reference). Pre-adipocytes derived from subcutaneous white adipose tissue can also be differentiated into brown adipocytes by transfecting the pre-adipocytes with a coactivator of PPARγ called PPARγ coactivator 1α (PGC-1α). See, e.g., Tiraby, et al., J. Biol. Chem., 278: 33370-33376, 2003, which is incorporated herein by reference. Adenovirus-mediated expression of PGC-1α increases the expression of UCP1, respiratory chain proteins, and fatty acid oxidation enzymes in human subcutaneous white adipocytes, while other changes in expression are consistent with that of brown adipocyte mRNA expression profile. In some instances, brown adipocytes can be derived from pre-adipocytes isolated from brown adipose tissue and immortalized using a viral promoter, e.g., SV40 T oncogene promoter. See, e.g., U.S. Pat. No. 6,071,747, which is incorporated herein by reference.

Brown adipocytes for transplantation into a neonatal subject can be derived from differentiation of stem cells or progenitor cells. Stem cells can include, but are not limited to, embryonic stem cells (including cells from embryonic stem cell lines), adult stem cells, induced pluripotent stem cells, mesenchymal stem cells, totipotent stem cells, multipotent stem cells, pluripotent stem cells, adipose-derived stem cells, muscle-derived stem cells, or bone-marrow derived stem cells. In general a stem cell refers to a cell having the capacity to self-renew and to differentiate into mature, specialized cells. A totipotent stem cell can differentiate into any type of body cell plus all of the extraembryonic cell types. A pluripotent stem cell can differentiate into to any type of body cell, and a multipotent stem cell can differentiate into multiple cell types of a particular tissue, organ, or physiological system. An example of pluripotent stem cells includes, but is not limited to, embryonic stem cells. An example of multipotent stem cells includes hematopoietic stem cells that can differentiate into any blood cell type. Mesenchymal stem cells are multipotent stem cells that can be differentiated into a variety of cell types, including adipocytes. Mesenchymal cells can be isolated from adipose tissue as well as from bone marrow, skeletal muscle, umbilical cord blood, placenta, and others. See, e.g., WO2006/051538; Elabd, et al., Stem Cells 27: 2753-2760, 2009; Lee, et al., Blood, 103:1669-1675, 2004; and Gomillion & Burg, Biomaterials, 27: 6052-6063, 2006; which are incorporated herein by reference.

The stem or progenitor cells for differentiation into brown adipocytes can be autologous cells derived from the neonatal subject including, but not limited to adipose-derived stem cells, cord blood stem cells, placental stem cells, muscle-derived stem cells, or bone marrow-derived stem cells isolated from neonatal subject-associated tissue, e.g., bone marrow, adipose, cord blood, placenta, or skeletal muscle. For example, multipotent adipose-derived stem (MADS) cells, which have high proliferative and differentation capabilities, can be obtained from harvested adipose tissue, e.g., from a biopsy of adipose tissue of the neonatal mammalian subject or of an allogeneic donor. MADS cells can be isolated from the stromal vascular fraction of collagenase-treated adipose tissue using adherence properties. MADS cells can be expanded in vitro by culturing in the presence of Fibroblast Growth Factor 2 (FGF2). See, e.g., Rodriguez J Exp Med. 201: 1397-1405, 2005. MADS can be differentiated into brown adipocytes using differentiation medium containing insulin, isobutyl methylxanthine, dexamethasone, and rosiglitazone. See, e.g., Elabd, et al., Stem Cells 27: 2753-2760, 2009, which is incorporated herein by reference.

Exogenous brown adipocytes for transplantation into a neonatal mammalian subject can be derived from stem cells or progenitor cells isolated from adipose or nonadipose tissue isolated from an autologous tissue or from allogeneic donor tissue. Tissue or cell sources from which to derive stem cells or progenitor cells that are capable of developing into mature adipocytes include, but are not limited to, bone marrow, muscle tissue, adipose tissue, umbilical tissue, embryonic stem cells, adult stem cells, or stem cell lines. For example, bone marrow contains mesenchymal stem cells, which can be differentiated into adipocytes. Bone marrow can be isolated by aspiration, and a density gradient can be used to isolate a small percentage of mesenchymal stem cells. These cells will proliferate until exposed to culture medium containing differentiation factors. Adipogenic differentiation of mesenchymal cell cultures derived from bone marrow can be induced to form adipocytes by treatment with isobutyl methylxanthine, dexamethasone, insulin, and indomethacin. See, e.g., Pittenger, et al., Science, 284: 143-147, 1999, which is incorporated herein by reference. In some protocols for differentiation of mesenchymal stem cells isolated from adipose, following growth to confluence, 20 nM to 100 nM rosiglitazone is added to culture medium and dexamethasone and isobutyl methylxanthine are omitted. See, e.g., Elabd, et al., Stem Cells 27: 2753-2760, 2009, which is incorporated herein by reference. Similar methods have been described for generating adipocytes from progenitor cells isolated from adipose, cord-blood, placenta, or skeletal muscle. See, e.g., Kang, et al., Cell Biol. Int. 30: 569-575, 2006; Park, et al., Cell Metab., 8: 454-457, 2008; Seale, et al., Nature, 454: 961-968, 2008; and WO2006/051538, which are incorporated herein by reference.

Brown adipocytes for transplantation into a neonatal mammalian subject can be derived from stem cells or from myoblasts isolated from fetal, neonatal, or mature skeletal muscle. Brown adipocytes and myocytes appear to share a common precursor, specifically a cell lineage that expresses the Myf5 gene. See, e.g., Seale, et al., Genes Dev. 23: 788-797, 2009, which is incorporated herein by reference. Brown adipocytes for use in a neonatal subject can be derived from CD34⁺ (CD146⁻, CD45⁻, & CD56⁻) progenitor cells isolated by digestion and immunoselection/depletion from skeletal muscle. See, e.g., Crisan, et al., Stem Cells, 26: 2425-2433, 2008, which is incorporated herein by reference. Brown adipocytes for transplantation can also be derived from skeletal muscle myoblast cell lines. For example, ectopic expression of PRDM16 in primary or established committed myoblasts causes the cells to adopt a brown adipocyte phenotype when exposed to pro-adipogenic stimuli. See, e.g., Seale, et al., Nature 454: 961-967, 2008, which is incorporated herein by reference. Brown adipocytes for use in a neonatal subject can be derived from multipotent stem cells or myoblasts isolated from enzyme-dissociated muscle tissue. See, e.g., Asakura et al., Differentiation 68: 245-253, 2001, which is incorporated herein by reference. Myoblasts for use in differentiation into brown adipocytes for use in transplantation into human neonatal subjects can be can be derived from biopsy or resection of skeletal muscle. See, e.g., Baj, et al., J. Translational Med. 3 (21): 2005 and De Coppi, et al., Diabetologia, 49 (8): 1962-73, 2006, which are incorporated herein by reference. In some instances, myoblast cell lines are available from a commercial source, examples of which include, but are not limited to, C2C12, H9c2(2-1), G-7, L6, A-10, L8, So18, and LHCN-M2 (e.g., from American Type Culture Collection (ATCC), Manassas, Va.).

Brown adipocytes for transplantation into a neonatal mammalian subject can be derived from differentiation of established tissue culture cell lines. Examples of mouse tissue culture cell lines that are capable of differentiation into adipose cells include, but are not limited to, 3T3-L1 cells and 3T3-F442A cells, RBM-Ad cells, C3H10T1/2 cells, 7F2 cells, D1 cells, HB2 cells, FVB cells, BFC-1 cells, and HIB 1B cells. See, e.g., Klein, et al., BioEssay, 24: 382-388, 2002, which is incorporated herein by reference. Examples of human cell lines that are capable of differentiation into adipose cells include, but are not limited to, human embryonic stem cell (hESC), PAZ6 cells, and prostate cell lines. See, e.g., Strom, et al., Human Reproduction, 22 (12): 3051-3058, 2007; Rajala, et al., PLoS One. 5 (4): e10246, 2010; Gomillion & Burg, Biomaterials, 27: 6052-6063, 2006; Mahmoud, et al., J Bone Miner Res. 25 (6): 1216-33, 2010; Zhau, et al., Clin Cancer Res. 17 (8): 2159-69, 2011; and Zilberfarb, et al., Journal of Cell Science 110: 801-807,1997; which are incorporated here by reference.

Prior to differentiation and/or implantation pre-adipocytes, precursors, progenitor cells, or stem cells, including cells of established cell lines, can undergo proliferation and expansion, either in vitro or in vivo. In vitro culture methods can be used in which the pre-adipocytes, precursors, progenitor cells, or stem cells are cultured in a growth medium that allows for proliferation and expansion of the cells. A growth medium containing, e.g., buffer, nutrients, amino acids, salts, and glucose (available commercially as various basal media, e.g., KnockOut™ D-MEM or KnockOut™ D-MEM/F12 basal media from Invitrogen, Carlsbad, Calif.) can be supplemented with antibiotics/antimycotics (e.g., penicillin, streptomycin, or amphotericiri B) and with serum, e.g., pathogen free fetal bovine serum, autologous serum, or commercially available allogeneic human serum. As an alternative to serum, serum-free or xeno-free supplements can be included in cell growth medium. Formulations for serum-free or xeno-free supplements have been published (see, e.g. Rajala, et al., PLoS One. 5 (4): e10246, 2010.) Serum-free or xeno-free supplements commercially available include, e.g., KnockoutTM serum replacement (KO-SR) serum-free (SF) supplement or KnockOutTM SR xeno-free (XF) supplement, both from Invitrogen, and Biopure™ Human Serum Albumin (HSA) from Biological Industries, Kibbutz Beit-Haemek, 25115, Israel. Serum-free or xeno-free complete media are commercially available and include, e.g., STEMPRO® hESC serum free medium, STEMPRO® MSC serum free medium, and KnockOut™ SR XF complete medium, all from Invitrogen, and HEScGRO animal component-free medium (Millipore; Billerica, Mass.), TeSR®1 and TeSR®2 defined, feeder-independent maintenance medium for hESCs and hiPSCs (StemCell Technologies, Vancouver, BC, Canada), and AF NutriStem™ hESC XF medium for human ES & iPS cell culture (Biological Industries). See, e.g., Rajala, et al., PLoS One. 5 (4): e10246, 2010; Hartmann et al., J Immunol Methods, 363 (1): 80-89, 2010; and Lund, et al., Gytotherapy, 11: 189-197, 2009, which are incorporated herein by reference. The growth culture can include feeder cells, including xenogeneic or allogeneic feeder cells. A variety of feeder cells are available from ATCC (Manassas, Va.). Growth culture conditions can include feeder cell-free conditions. The growth medium can include growth factors for use in promoting and enhancing proliferation and/or inhibiting differentiation. The growth medium may include fibroblastic growth factor 2 (FGF-2). See e.g. Rajala, et al., PLoS One. 5 (4): e10246, 2010, and Zaragosi et al., Stem Cells 24: 2412-2419, 2006, which are incorporated herein by reference. Other factors that may be added to medium to promote growth or differentiation can include but are not limited to thyroid hormone, epidermal growth factor (EGF), transforming growth factor-β (TGF-β), and platelet-derived growth factor (PDGF). For example, human MADS cells can be grown in a basal medium of Dulbecco's modified Eagle's medium (DMEM) supplemented with serum or its equivalent, FGF-2, and penicillin/streptomycin. For example, progenitor cells such as those derived from umbilical cord blood can be grown in Iscove modified Dulbecco medium, supplemented with 10% serum and penicillin/streptomycin. See, e.g., Invitrogen, Carlbad, Calif. See, e.g., Kang, et al, Cell Biol. Int. 30: 569-575, 2006, which is incorporated herein by reference. For example, hES or induced pluripotent stem cells (iPSC) can be cultured and expanded in medium containing KO-DMEM basal medium supplemented with 20% KO-SR, 2 mM Glutamax, 0.1 mM β mercaptoethanol, 0.1 mM MEM non-essential amino acids (NEAA), 1% penicillin-streptomycin, and 8 ng ml/ml recombinant human bFGF. For example, human adipose-derived stem cells (ASC) can be cultured and expanded in DMEM/F-12 basal media, supplemented with 1% GlutaMAX, 1% antibiotics/antimycotics (a/a; 100 U/mL penicillin, 0.1 mg/mL streptomycin, and 0.25 mg/mL amphotericin B) and 10% alloHS (PAA Laboratories GmbH, Pasching, Austria). For example, human umbilical cord-derived stem cells can be cultured and expanded under xeno-free (XF, containing GMP-certified human serum) or serum-free (SF) culture conditions. See, e.g., Hartmann et al., J Immunol Methods, 363 (1): 80-89, 2010, which is incorporated herein by reference. For example, hESC, iPSC, or ASC can be cultured and expanded in (RegES) xeno-free medium comprising knockout-Dulbecco's modified Eagle's medium (KO-DMEM base supplemented with human serum albumin, amino acids, vitamins, antioxidants, trace minerals, and growth factors. See, e.g., Rajala, et al., PLoS One. 5 (4): e10246, 2010, which is incorporated herein by reference. The cells can be grown in tissue culture plates or flasks and maintained at 37° C. in 5% CO₂ in a fully humidified incubator.

Pre-adipocytes, precursors, stem cells, or progenitor cells, including those isolated from the neonatal subject-associated tissue, allogeneic donor-associated tissue, or established or primary cell lines can be expanded, and appropriate differentiation factors added to induce differentiation of the cells into brown adipocytes. The stem cells can be administered to the neonatal subject at any cell stage and can be matured or differentiated either in vitro or in vivo. The differentiation of pre-adipocytes or progenitor stem cells into brown adipocytes can include stimulation by one or more growth factors, differentiation factors, or transcriptional factors. In general, pre-adipocytes or progenitor cells, or stem cells are differentiated into adipocytes by exposure to pro-adipogenic stimuli. In vitro, the pro-adipogenic stimulus can be a cocktail of reagents added to the cell culture medium and can include one or more of 3-isobutyl-1-methylxanthine, dexamethasone, dibutyryl cyclic AMP, insulin, transferrin, triodothyronine, rosiglitazone, and indomethacin. Factors can also be used to promote differentiation of the adipocyte lineage. For example, one or more bone morphogenetic protein (BMP), such as BMP2, BMP4, BMP6 or BMP7 can be added to the culture to promote differentiation into brown adipocytes. In particular BMP7 can be added to the culture medium. When BMP7 is added in culture to brown pre-adipocytes isolated from adipose tissue, the expression of UCP-1 increases, as does the expression of several regulators of adipocyte differentiation including PRDM16, PGC-1α, and PGC-1β. In addition, BMP7 induces an increase in mitochondrial density, consistent with the phenotype of differentiated brown adipocytes. Similarly, the addition of BMP7 to multipotent stem cells, e.g., C3H10T1/2 cells, results in a mature brown adipocyte phenotype with marked increases in lipid accumulation and induction of UCP-1. See, e.g., U.S. Pat. No. 7,576,052 and Tseng, et al., Nature, 454: 1000-1004, 2008, which are incorporated herein by reference.

In general, the nuclear hormone receptor peroxisome proliferator-activated receptor γ (PPARγ) appears to be important for development of brown adipose tissue. For example, PPARγ-deficient mice (PPARγ-/-) do not develop normal brown adipose tissue (BAT) deposits and die shortly at or before gestational day 12.5. The activity of PPARγ can be stimulated by a variety of ligands including, but not limited to, natural eicosanoid 15-deoxy-Δ^{12,14}-prostaglandin J₂, endogenous constituents of oxidized low density lipoprotein (oxLDL) particles, e.g., 9- and 13-HODE, perfluorononanioc acid, and synthetic thiazolidinedione (TZD) compounds, e.g., rosiglitazone, pioglitazone, rivoglitazone, and ciglitazone. Including one or more PPARγ agonist in the culture medium can aide differentiation of precursor cells into adipocytes. See, e.g., Barak, et al., Molecular Cell, 4: 585-595, 1999, which is incorporated herein by reference.

The differentiation of pre-adipocytes, precursors, progenitor cells, or stem cells into brown adipocytes can also be stimulated by treatment with one or more transcription factors. Examples of transcription factors involved in adipocyte differentiation include, but are not limited to, RBI, p107, RBL1, RIP 140, NRIP1, FOXC2, PRDM16, GATA2. For example, PRDM16 (“PR domain containing 16”) is a transcriptional co-regulatory protein and contributes to brown adipocyte differentiation by co-activating PPARy, the latter of which is considered the master gene of adipocyte differentiation. PRDM16 can also complex with other DNA-binding factors, e.g., C/ERP-β to initiate brown adipocyte differentiation. See, e.g., Seale et al., Genes Dev., 23: 788-797, 2009; Cannon & Nedergaard, Physiol. Rev., 84: 277-359, 2004; and U.S. Patent Application 2011/0059051, which are incorporated herein by reference.

Pre-adipocytes, precursors, progenitor cells, or stem cells can be differentiated into brown adipocytes in vitro in the presence of a pro-adipogenic reagent mix, components of which can include, but are not limited to isobutyl methylxanthine, dexamethasone, dibutyryl cyclic AMP, insulin, transferrin, triodothyronine, rosiglitazone, indomethacin and ascorbic acid. When the cells have reached an appropriate cell number and confluency (e.g., 70-95% confluence), a pro-adipogenic reagent mix is added to induce terminal differentiation of the cells into brown adipocytes. After about 3 to 30 days in pro-adipogenic reagent mix, the cells can be assessed for phenotypes characteristic of brown adipocytes using one or more assays. Assays for assessing phenotypes characteristic of brown adipocytes include, but are not limited to, staining with Oil-red O to detect lipid droplets, polymerase chain reaction (PCR) amplification, Northern blot analysis, and/or Western blot analysis to detect expression of brown adipocyte specific transcripts and proteins (e.g., UCP-1), and microscopy to detect increased mitochondrial numbers. See, e.g., U.S. Application 2009/0054487 and PCT Application WO/2005/123049A2, which are incorporated herein by reference.

Differentiation of pre-adipocytes, progenitor cells, or stem cells into brown adipocytes can be facilitated by genetically modifying the cells to express factors known to promote brown adipocyte differentiation. Gene transfer techniques are known by persons of ordinary skill in the art, and can include viral and non-viral transfection techniques. See, e.g., Verma, et al., Gene Therapy 5: 692-699, 1998; and Papapetrou, et al., Gene Therapy 12: S118-S130, 2005, which are incorporated herein by reference. Inducing PRDM 16 gene expression in adipocytes can induce brown adipocyte differentiation in the mammalian subject. Increasedbrown fat differentiation in the mammalian subject can induce the expression of mitochondrial genes and cellular respiration leading to a reduction in obesity in the mammalian subject. See, e.g., U.S. Patent Application 2011/0059051; PCT WO 2005/123049A2, which are incorporated herein by reference. For example, forced expression of the zinc-finger protein PRDM16 or the transcription factor C/EBP-13 in stromal-vascular stem cells isolated from white adipose tissue or in naïve fibroblastic cells using retroviral gene transfer vectors induces a brown adipocyte phenotype as exemplified by upregulation of UCP-1 and other markers of brown adipocyte differentiation including PGC1α, Cox8b, elov13, Adipoq, and Adipsin. Transplanting cells modified with PRDM16 and C/EBP-β into a mammalian subject results in depots of functional brown adipose tissue as exemplified by a positive response to ¹⁸F-fluorodeoxyglucose computed tomography. Ectopic over-expression of UCP-1 in pre-adipocytes has also been described. See, e.g., Seale, et al., Cell Metab., 6: 38-54, 2007; and Kajimura, et al., Nature 460: 1154-1158, 2009; Si, et al., J. Lipid Res., 48: 826-838, 2007, which are incorporated herein by reference.

The brown adipocytes for transplantation into a neonatal subject can be modified to include genetic modifications that change, enhance, or supplement the function of the transplanted cells. The genetic modifications can include expression of gene products that promote differentiation of the pre-adipocytes, precursors, progenitor cells, or stem cells into metabolically active brown adipocytes. The genetic modifications can include expression of gene products that can promote the vascularization of the tissue comprising the transplanted brown adipocytes. Examples of gene products that can promote the vascularization of the tissue comprising the transplanted brown adipocytes include VEGF and other known angiogenic factors. Similarly, the genetic modifications can include expression of modulators of brown adipocyte function. Examples of modulators of brown adipocyte function include BMP7 or activators of β adrenergic receptors. In an aspect, genetic modification to change, enhance, or supplement the function of the transplanted brown adipocytes can be made to non-adipocytes that are configured to express growth and/or differentiation factors and are co-transplanted with the brown adipocytes.

The brown adipocytes for transplantation into a neonatal mammalian subject can be modified to include a marker to enable tracking of the cells following transplantation to determine viability and sustainability of the transplanted cells. The marker can be one or more of a fluorescent marker, a magnetic marker, an RFID tag, a radioactive marker, a radiopaque marker or a combination thereof that can be used to monitor the transplanted cells. In some instances, the marker can be genetically incorporated into the brown adipocytes, for example, a green fluorescent protein marker. More generally, the increase in brown adipocytes represented by the transplanted cells can be reflected in increased glucose metabolism as monitored by positron-emission tomography (PET) and computed tomography (CT) in the presence of ¹⁸F-fluorodeoxyglucose as described by Virtanen, et al., (in N. Engl. J. Med. 360: 1518-1525, 2009, which is incorporated herein by reference). Alternatively, the activity of brown adipose tissue in neonates can be assessed using gamma-camera imaging with ^{99m}Tc-tetrofosmin. See, e.g., Fukuchi, et al., J. Nucl. Med., 44: 1582-1585, 2003, which is incorporated herein by reference.

The brown adipocytes for transplantation into a neonatal mammalian subject can be identified and further regulated by regulating expression of uncoupling protein 1 (UCP1) in the brown adipocytes. The brown adipocytes for transplantation into a neonatal mammalian subject can be identified by expression of UCP1 as a marker. UCP1 is an important component of non-shivering thermogenesis mediated by brown adipose tissue. UCP1 is also a specific marker of differentiation of pre-adipocytes and progenitor cells into brown adipocytes. UCP1 is a 32 kDa protein expressed in the inner membrane of the mitochondria. UCP1 allows the dissipation of the proton electrochemical gradient generated by the respiratory chain. Uncoupling between oxygen consumption and ATP synthesis promotes energy dissipation as heat. Fatty acids and retinoids have been shown to activate UCP1. In neonatal mammals, hibernators and rodents, cold-induced thermogenesis in brown adipose tissue (BAT) contributes to the maintenance of body temperature. Fuel is provided as fatty acids derived from brown adipocyte and white adipocyte lipolysis. UCP1 biosynthesis is mainly controlled at the transcriptional level. During cold exposure, for example, sympathetic nervous system stimulation of brown adipose tissue is the primary signal that activates UCP1 gene expression.

The transcriptional regulation of UCP1 expression is controlled by a number of regulatory elements including, but not limited to, peroxisome proliferator-activated receptor (PPAR), CCAAT/enhancer-binding protein (C/EBP) families and cAMP response-binding protein (CREB). UCP1 expression, is also regulated by an adrenergic signaling mechanism in which G-protein receptor mechanism coupling cAMP production to protein kinase A (PKA)-dependent phosphorylation of CREB and p38 MAP kinase. UCP-1 expression can be upregulated in the presence of a cAMP phosphodiesterase inhibitor (e.g., caffeine), β adrenergic receptor agonist (e.g., propranolol), or a PPARγ agonist (e.g., rosiglitazone). Retinoic acid and thyroid hormones are other positive regulators of UCP-1 expression. See, e.g., Cannon & Nedergaard Physiol. Rev. 84: 277-359, 2004 and Kozak and Koza, Int. J. Obes. 32 (Suppl 7): S32-S38, 2008, which are incorporated herein by reference.

### Methods For Administration of A Therapeutic Composition Including Brown Adipocytes

A method for modulating heat loss in a neonatal mammalian subject is described herein that includes providing exogenous brown adipocytes, or precursors thereof, to an internal site in the neonatal mammalian subject. The method further includes providing the brown adipocytes to an internal site including, but not limited to, a subcutaneous site, a scapular site, an axillary site, a thoracic site, an abdominal site, or blood vessel site of the neonatal mammalian subject. The brown adipocytes can be provided to a neonatal mammalian subject by implantation and/or by injection. Implantation can be achieved by injection.

The brown adipocytes can be provided to a neonatal mammalian subject by injection, for example, by injecting into a subcutaneous site, an intraperitoneal site, intramuscular site, or vascular site. The brown adipocytes can be administered to a neonatal mammalian subject, e.g., by injection, as a cell suspension in a pharmaceutically acceptable carrier. In an aspect, the pharmaceutically acceptable carrier is a suspension fluid, for example, normal saline (0.91% w/v sodium chloride). Other suitable suspension fluids for cells or tissues include, but are not limited to, lactated Ringer's solution or serum-free culture medium with or without additives including, but not limited to, heparin, insulin, vitamin E, and nonsteroidal anabolic hormones. The suspension fluid can further include one or more of the medicaments described herein to promote the growth and viability of the brown adipocytes or surrounding tissue or affect function thereof.

The brown adipocytes can be provided to a neonatal mammalian subject, e.g. by implantation, including surgical implantation, and/or by injection, into, e.g., a subcutaneous site, intramuscular site, intraperitoneal site or vascular site. For example, the brown adipocytes or precursors thereof, can be adhered to, encapsulated in, and/or cultured on one or more solid or semi-solid biocompatible carrier suitable for implantation and/or injection into the neonatal mammalian subject. In an aspect, the biocompatible carrier for providing brown adipocytes is one or more of a membrane, a natural matrix, a synthetic matrix, woven or non-woven fiber based matrix, a hydrogel, a natural sponge, a synthetic sponge, microbeads, macroparticles, microparticles, or microspheres and/or in an encapsulating material. See, e.g., P. Bauer-Kreisel et al. Cell-delivery therapeutics for adipose tissue regeneration, Advanced Drug Delivery Reviews 62: 798-813, 2010; Zomillion & Burg, Biomaterials 27: 6052-6063, 2006; Patrick, Anat. Rec. 263: 361-366, 2001, and Morgana, et al., Formation of a human-derived fat tissue layer in PDLLGA hollow fibre scaffolds for adipocyte tissue engineering; Biomaterials 30 (10): 1910-1917, 2009, See, e.g., Hong et al., Adipose Tissue Engineering by Human Adipose-Derived Stromal Cells, Cells Tissues Organs 183: 133-140, 2006, which are incorporated herein by reference.

The brown adipocytes can be provided to a neonatal mammalian subject e.g. by implantation and/or by injection in a formulation that includes a hydrogel. Hydrogels can be used as a growth matrix for pre-adipocytes, progenitor cells, stem cells, and/or partially or fully differentiated brown adipocytes. Cell culture-incorporating and/or cell-encapsulating hydrogels can be used as injectable cell carriers. Examples of hydrogels for use providing brown adipocytes include, but are not limited to, alginate, hyaluronic acid, polyethylene glycol (PEG), and/or fibrin gels. The hydrogel gelation can be controlled by temperature or chemical means: In an aspect, brown adipocytes can be combined with a hydrogel just prior to injection, in a form encapsulating the cells, as described herein. Brown adipocytes can be homogenously mixed in a hydrogel in its liquid state and subsequently injected as a gel state. The brown adipocytes can be homogenously mixed in a temperature-sensitive hydrogel in its liquid state at room temperature (22-25° C.) and allowed to gel upon reaching body temperature (e.g., 37° C., after subcutaneous injection). Alternatively or in addition, brown adipocytes and their precursors can be cultured on hydrogels, and can be expanded and/or induced to differentiate prior to injection.

Certain hydrogels, including some available from commercial sources, are derivatives of extracellular and/or basement membrane products. BD Matrigel™ Matrix and BD™ Laminin/Entactin Complex are available from Becton Dickinson have been used for culturing human embryonic stem cells and induced pluripotent stem cells, and can potentiate in vivo adipogenesis of exogenously applied pre-adipocytes in mammals. Myogel, available from Biolink in Australia, is derived from an muscle tissue, and adipose protein-derived and dermis-derived gels that can sustain adipogenesis have also been established and investigated in vivo. Fibrin hydrogels encapsulating adipose-derived stem cells have been successfully implanted in mammals into both subcutaneous and intramuscular tissues. Polyethylene glycol (PEG) is a synthetic hydrogel commonly used for tissue engineering. Stem cells, including cells adipogenically induced, have successfully been implanted in mammalian subcutaneous tissues when encapsulated in PEG-di(meth)acrylate (PEGDA) hydrogel. These synthetic hydrogels can be modified with peptides to promote cell adhesion. See Bauer-Kreisel, et al., Cell-delivery therapeutics for adipose tissue regeneration, Advanced Drug Delivery Reviews 62: 798-813, 2010.

A scaffold, whether natural or synthetic, refers to a three-dimensional template for colonization by pre-adipocytes, progenitor cells, stem cells, and/or partially or fully differentiated brown adipocytes. Colonization can take place in vitro, e.g. in a cell culture, or in vivo, e.g., when the scaffold is implanted and exogenous cells provided separately. In an aspect, the scaffold can include one or more biodegradable polymers. Examples of natural polymers for use in a three-dimensional matrix include, but are not limited to, polypeptides, e.g., albumin, fibrinogen, fibrin, collagen and gelatin, hyaluronic acid, and also polysaccharides, e.g., chitin, chitosan, alginate and agarose. These natural polymers can also be modified, where appropriate; for example, proteins such as collagen can be crosslinked. Examples of synthetic polymers for use in a three-dimensional matrix include, but are not limited to, polyanhydrides, e.g., poly(sebacic acid-hexadecanoic diacid), poly(ε-caprolactone), poly(orthoesters), (polytetrafluoroethylene), polyethylene glycol (PEG), and, especially, poly(α-hydroxy-esters), e.g., poly(glycolic acid), poly(lactic acid), poly(glycolic acid-lactic acid) (PGA/PLA/PLGA). Scaffolds can be designed in a variety of geometric shapes and with defined physical properties including porosity, diameter and surface area.

In an aspect, a scaffold can be designed to be soft and non-rigid with optimum surface area, for example, using a natural material to form a sponge having a certain size pore or a synthetic polymer forming a high surface area fiber. Pre-adipocytes, precursors, progenitor cells, stem cells, and/or partially or fully differentiated brown adipocytes can be seeded onto a soft biocompatible scaffold, with or without encapsulation, and can be provided to a neonatal mammalian subject, e.g. by implantation in a subcutaneous site. The biocompatible scaffold can be seeded and colonized ex vivo prior to implantation and/or can be implanted/injected into the neonatal subject and seeded in situ. For example, a collagen or gelatin sponge, e.g. having a pore size of 200-400 micrometers, may be seeded with pre-adipocytes, which are then expanded and differentiated in vitro. The gelatin sponges carrying the cells can be implanted into subcutaneous tissue of the abdomen of the neonatal mammalian subject following a small incision. A porous gelatin sponge is available commercially (Gelfoam, Pharmacia & Upjohn, Kalamazoo, Mich., USA) and has been used extensively in clinical and tissue engineering studies as a scaffold. Adipose-derived stem cells grown on the gelatin sponge, and induced to differentiate, then subcutaneously implanted in an adult mammal. See, e.g., Hong et al., Adipose Tissue Engineering by Human Adipose-Derived Stromal Cells, Cells Tissues Organs, 183: 133-140, 2006.

The biodegradable scaffold can be in various forms that include both injectable and surgically implantable designs. A scaffold may be designed with a particular geometric shape and/or with defined physical properties including porosity, diameter and surface area. Pre-adipocytes, precursors, progenitor cells, stem cells, and/or partially or fully differentiated brown adipocytes can be seeded onto a scaffold formed by synthetic biocompatible material that is a porous biodegradable polymer e.g., poly (L-lactic-co-glycolic) acid (PLGA), with or without encapsulation. For example, a number of different scaffolds of PLGA and related polymers have been shown to sustain stem cells, including adipose-derived stem cells, and to promote adipogenesis. Designs include fiber meshes, electrospun nanofibrous scaffolds, and hollow fiber scaffolds. In vivo adipogenesis has been demonstrated with PLGA scaffolds seeded with adipose-derived stem cells. Likewise, PLGA hollow fiber scaffolds seeded with human bone marrow stem cells formed 300 micrometer thin layers of adipocyte-containing tissue; these tissue-scaffolds were encapsulated within alginate/chitosan hydrogel capsules and subcutaneously implanted in an adult mammal. See, e.g., Goren et al., Encapsulated human mesenchymal stem cells: a unique hypoimmunogenic platform for long-term cellular therapy, FASEB Journal 24: 22-31, 2010; P. Bauer-Kreisel et al., Advanced Drug Delivery Reviews 62: 798-813, 2010; Morgana, et al., Formation of a human-derived fat tissue layer in PDLLGA hollow fibre scaffolds for adipocyte tissue engineering, Biomaterials 30 (10): 1910-1917, 2009. Other materials used in scaffolds include, but are not limited to, collagen, fibrin, gelatin microspheres,,and natural sponge. See, e.g., Bauer-Kreisel, et al., Advanced Drug Delivery Reviews 62: 798-813, 2010; Gomillion & Burg, Biomaterials 27:6052-6063, 2006; Roberts, et al., Eur. Cells Materials, 16:89, 2008; U.S. Pat. Applications 2008/0286241, 2010/0015104, and 2010/0028405; Patrick Anat. Rec., 263: 361-366, 2001, which are incorporated herein by reference.

The brown adipocytes can be provided to an internal site of the neonatal mammalian subject, e.g. by implantation and/or by injection, in a formulation that includes microbeads, microcapsules, microparticles, or microspheres. Cells can be adhered to or cultured on solid or porous microspheres or microparticles that can then be provided to the neonatal subject, either alone or in combination with a hydrogel. For example, cells can be precultured on poly(lactic-co-glycolic) acid (PLGA) microspheres as the cell carrier, e.g., including adipogenic induction, and still have the carrier in an injectable form. See, e.g., P. Bauer-Kreisel et al. Advanced Drug Delivery Reviews 62: 798-813, 2010. For example, microparticles constructed from crosslinked chitosan can be used as an_{:}injectable platform for tissue engineering. See, e.g., Cruz, et al., Chitosan microparticles as injectable scaffolds for tissue engineering, J. Tissue Eng. Regen. Med. 2: 378-380, 2008, which is incorporated herein by reference. Microparticles or microspheres can be generated using one or more of the natural or synthetic polymers described herein, examples of which include, but are not limited to, albumin, fibrinogen, collagen, gelatin, chitin, chitosan, alginate, agarose, poly(sebacic acid-hexadecanoic diacid), poly(ε-caprolactone), poly(orthoesters), poly(glycolic acid), poly(lactic acid) or poly(glycolic acid-lactic acid), or may be derived from tissue, including extracellular matrix powders. Microspheres or microparticles may also be modified to enhance better cell adhesion; for example, alginate beads can be modified with the cell adhesion peptide RGD.

The brown adipocytes can be provided to an internal site of the neonatal mammalian subject, e.g., by implantation, including surgical implantation into, e.g., a subcutaneous site, intramuscular site, intraperitoneal site or vascular site as a three dimensional tissue. For example, brown adipocytes from human adipose-derived stem cells can be cultured as a three dimensional sheets employing a “self-assembly” culture methodology, then harvested and provided to the neonatal human subject by subcutaneous implantation. See, e.g., P. Bauer-Kreisel et al. Advanced Drug Delivery Reviews 62: 798-813, 2010.

The matrix, scaffold, or microparticle/microsphere material can be designed to include chemical or surface modifications that enhance attachment, proliferation, differentiation, and/or sustainability of the brown adipocytes. For example, the material can include one or more adhesive molecules to promote cellular attachment and/or one or more growth factors or other medicaments described herein that promotes the growth or viability of the brown adipocytes. Examples of adhesive molecules to which cells are known to attach include one or more extracellular matrix proteins, e.g., collagen, laminin and fibronectin, as well as adherence-promoting peptides such as RGD. Examples of biologically active substances for surface modification of a natural or synthetic matrix, scaffold, or microparticle include, but are not limited to, synthetic active compounds (inorganic or organic molecules), proteins, polysaccharides and other sugars, lipids and nucleic acids which, for example, influence cell growth, cell migration, cell division, cell differentiation and/or tissue growth or possess therapeutic, prophylactic or diagnostic effects. Examples of biologically active substances for surface modification of a natural or synthetic matrix, scaffold, or microparticle can further include, but are not limited to, vasoactive active compounds, neuroactive active compounds, hormones, growth factors, cytokines, steroids, anticoagulants, anti-inflammatory active compounds, immunomodulating active compounds, cytotoxic active compounds, antibiotics and antiviral active compounds. The matrix, scaffold, or microparticle/microsphere material can include one or more factors for promoting growth, e.g., FGF. The material can include one or more factors for inducing differentiation, e.g., insulin and dexamethasone. See, e.g., Rubin, et al., Encapsulation of adipogenic factors to promote differentiation of adipose-derived stem cells, J Drug Target. 17 (3): 207-215, 2009 which is incorporated herein by reference

The matrix, scaffold, or microparticle/microsphere material can be further seeded with cells that promote vascularization, e.g., mature endothelial cells and/or vascular smooth muscle cells and/or progenitors thereof. One or more factors formulated to promote cell growth and/or angiogenesis, for example basic fibroblast growth factor (bFGF), acidic fibroblast growth factor, or vascular endothelial growth factor (VEGF), can be added to the matrix, scaffold, or microparticle/microsphere material.

Methods for modulating heat loss in a neonatal mammalian subject can include providing brown adipocytes for transplantation to an internal site of the neonatal mammalian subject. The brown adipocytes can be delivered, for example, by implantation and/or injection, as encapsulated brown adipocytes. In an aspect, encapsulation can provide immunoisolation of the brown adipocytes in the neonatal subject to prevent an immune response and rejection of the transplanted cells. Encapsulation can be particularly advantageous when the brown adipocytes are derived from an allogeneic or xenogeneic source. Immunoisolation can be achieved by encapsulating the cells, including the scaffolds or matrix on which they are grown, in a semi-permeable, biocompatible material that isolates the transplanted cells from attack by the neonatal subject's immune system but allows for exchange of nutrients, gases, and signaling molecules between the transplanted cells and the neonatal mammalian subject.

A variety of semipermeable polymeric and inorganic matrices and membranes with diverse physiochemical properties and geometries can be used to encapsulate the brown adipocytes and provide immune-isolation in the neonatal mammalian subject. Examples of encapsulating materials can include one or more membranes, natural matrix, synthetic matrix, macroparticles, microparticles, microspheres, macrocapsules, or microcapsules as described herein.

In an aspect, the brown adipocytes can be encapsulated in a natural matrix or synthetic matrix including an alginate. Alginates are a family of unbranched anionic polysaccharides originally derived from brown algae. Alginates are a linear polysaccharide copolymers with homopolymeric and heteropolymeric blocks of (1-4)-linked β-D-mannuronate (M) and α-L-guluronate (G) residues linked together in different sequences or blocks. In the presence of divalent cations, e.g., calcium, the alginate forms a biocompatible gelatin-like material stable at body temperature into which cells for transplantation can be encapsulated. See, e.g., U.S. Pat. Nos. 5,084,350 and 5,429,821; Ghidoni, et al., Cytotechnol. 58: 49-56, 2008, which are incorporated herein by reference. Encapsulation in alginate has been demonstrated to provide protection from an immune response for allogeneic or xenogenic transplantation of cells in a number of mammalian species including humans. See, e.g., Lim, et al., Cell microencapsulation, Adv Exp Med Biol. 670: 126-136, 2010, which is incorporated herein by reference. Encapsulation of stem cells in alginate, and specifically adipose-derived stem cells, has been described. See, e.g., Liu and Chang, Artificial cell microencapsulated stem cells in regenerative medicine, tissue engineering and cell therapy, Adv Exp Med BioL 670: 68-79, 2010 and U.S. Pat. No. 7,078,232, which are incorporated herein by reference.

Other materials for encapsulation of cells include, but are not limited to, agarose, poly(hydroxyethyl methacrylate-co-methyl methacrylate), polyethylene glycol-diacrylate, polyethylene glycol-lipid conjugates, polycations and anionic poly(styrene sulfonate), poly(diallyldimethylammonium chloride). See, e.g., Lim, et al., Adv Exp Med Biol. 670: 126-136, 2010, and Ghidoni, et al., Cytotechnol. 58: 49-56, 2008 which is incorporated herein by reference. In an aspect, encapsulation of the brown adipocytes can be achieved using an immunoisolation system that includes a mesh material. Examples of mesh materials used for this purpose include, but are not limited to, stainless steel mesh, polyethylene terephthalate mesh, and polytetrafluoroethylene mesh. Various angiogenic factors, e.g., basic fibroblast growth factor (bFGF), acidic fibroblast growth factor, and vascular endothelial growth factor (VEGF) can also be added as an adjunct to improve vascularization of the brown adipocyte transplant. See, e.g., Wilson & Chaikof, et al., Adv. Drug Deliv. Rev., 60: 124-145, 2008, which is incorporated herein by reference.

In an aspect, immunoisolation of the brown adipocytes from the neonatal immune system can be achieved using an immunoisolation system that includes a cage formed from a natural material such as one or more protein or lipid, or a synthetic material such as hydroxyapatite. An example includes structures composed of natural or synthetic bone-like material, e.g., hydroxyapatite, configured to provide living cells and tissues to subjects in a rigid or semi-rigid conformation. See, e.g., U.S. Patent Application 2008/0057095, which is incorporated herein by reference.

### Promotion of Angiogenesis In Brown Adipose Tissue

A method for modulating heat loss in a neonatal mammalian subject is described herein that includes providing exogenous brown adipocytes, or precursors thereof, to an internal site in the neonatal mammalian subject. The method can further include providing factors to the subject that promote adipose angiogenesis. Angiogenesis refers to the process by which new blood vessels are generated from existing vasculature and tissue. See, e.g., Folkman, J., Nat Med 1: 27-31, 1995, which is incorporated herein by reference. “Repair or remodeling” refers to the reformation of existing vasculature. The spontaneous growth of new blood vessels provides collateral circulation in and around an area of implanted brown adipocytes, improves blood flow, and alleviates the symptoms caused by ischemia. Angiogenic factor or angiogenic protein refers to any protein, peptide or other agent capable of promoting growth of new blood vessels from existing vasculature (“angiogenesis”). Suitable angiogenic factors for use to induce vascularization of brown adipose tissue include, but are not limited to, placenta growth factor, macrophage colony stimulating factor, granulocyte macrophage colony stimulating factor, vascular endothelial growth factor (VEGF)-A, VEGF-A, VEGF-B, VEGF-C, VEGF-D, VEGF-E, neuropilin, fibroblast growth factor (FGF)-1, FGF-2(bFGF), FGF-3, FGF-4, FGF-5, FGF-6, angiopoietin 1, angiopoietin 2, erythropoietin, BMP-2, BMP-4, BMP-7, TGF-β, IGF-1, osteopontin, pleiotropin, activin, endothelin-1, and combinations thereof. Angiogenic factors can act independently, or in combination with one another. When in combination, angiogenic factors can also act synergistically, whereby the combined effect of the factors is greater than the sum of the effects of the individual factors taken separately.

Angiogenic factors can be produced or obtained from any suitable source. For example, the factors can be purified from their native sources, or produced synthetically or by recombinant expression. Angiogenic factors are commercially available. The factors can be provided to the neonatal subject together with the brown adipocytes. For example, the factors can be provided with the cells in an injectable formulation. The factors may be provided with the cells as part of the growth matrix or scaffold, e.g., in a slow release formulation or as part of the modified matrix or scaffold material. The factors may be provided during culture, expansion, and/or differentiation. Instead or in addition, the factors can be administered to the neonatal subject directly, e.g. as a medicinal composition. Alternatively, the factors can be administered in the form of an expression plasmid encoding the factors. The construction of suitable expression plasmids is well known in the art. Suitable vectors for constructing expression plasmids include, for example, adenoviral vectors, retroviral vectors, adeno-associated viral vectors, RNA vectors, liposomes, cationic lipids, lentiviral vectors and transposons. See, e.g., U.S. Pat. No. 7,651,684 and U.S. Application 2010/00015104, which are incorporated herein by reference.

Alternatively or in addition, vascular structures can be pre-formed in vitro, for example in three dimensional cultures of adipocytes grown on scaffolds. For three-dimensional constructs of substantially large size, an early vascular supply throughout the whole composite can be a crucial requirement for adequate and homogeneous tissue development and long-term survival. Brown adipocytes cultured on scaffolds can include preformation of a capillary network by in vitro addition of endothelial cells and proangiogenic factors or addition of other engineered vascular constructs. See, e.g., Bauer-Kreisel et al. Advanced Drug Delivery Reviews 62: 798-813, 2010.

### Internal Sites For Transplantation of Brown Adipocytes

A method for modulating heat loss in a neonatal mammalian subject further includes providing the brown adipocytes to an internal site in the neonatal mammalian subject, including, but not limited to, a subcutaneous site, a subdermal site, a scapular site, an axillary site, a thoracic site, an abdominal site, or blood vessel site of the neonatal mammalian subject. Subcutaneous administration refers to administration to an area of the mammalian anatomy located beneath the skin or beneath the dermis. Administration to a scapular site refers to administration to an area of the mammalian anatomy proximal to the scapula or shoulder blade. A scapular site can further include an interscapular region or subscapular region. Administration to an interscapular region refers to administration to an area of the mammalian anatomy between the scapulae on the upper back. Administration to a subscapular region refers to administration to an area of the mammalian anatomy situated below or on the underside of the scapula. Administration to an axillary site refers to administration to an area of the mammalian anatomy under the arm, e.g., in the area of the armpit. Administration to a thoracic site refers to administration to an area of the mammalian anatomy pertaining to the thorax or that region of the body that lies between the head and the abdomen and includes the thoracic cavity containing the heart and lungs. Administration to an abdominal site refers to administration to an area of the mammalian anatomy that lies between the thorax and the pelvis and includes the abdominal cavity containing organs of the digestive and urinary systems. Administration to a blood vessel site refers to administration to an area of the mammalian anatomy that is either in or proximal to a blood vessel or a capillary bed. Administration to an internal site can further include administration to the supraclavicular region which refers to an area of the mammalian anatomy situated above the clavicle. Administration can include implantation or injection; implantation may be achieved by injection. Administration may be described as transplantation or implantation.

### Medicaments Administered In Combination With Transplanted Brown Adipocytes

Medicaments can be administered to the neonatal subject in combination with the transplanted brown adipocytes to further promote thermogenic activity of the brown adipocytes. Examples of medicaments that can be used to increase the thermogenic activity of brown adipocytes include, but are not limited to, one or more of a norepinephrine, β-adrenergic receptor agonist, PPAR agonist, NPY antagonist, uncoupling protein activating agents, thyroxine, serotonin reuptake inhibitor, MCH antagonist, GLP-1 agonist, 5-HT2c agonist, 5-HT2A agonist, galanin antagonist, CRF agonist, urocortin agonist, melanocortin agonist, enterostatin agonist, or orphosphodiesterase inhibitor.

Examples of medicaments that can be used to modulate heat loss in the neonatal mammalian subject and to modulate the thermogenic activity of brown adipocytes include, but are not limited to, β3 adrenergic receptor agonists, β3 adrenergic receptor trans-activating factor agonists, NPY1 antagonists, NPY4 antagonists, leptin agonists, Aryl alkyl acid derivatives and uncoupling protein (“UCP”) activating agents, specifically UCP1, UCP2 and/or UCP3 activating agents. See, e.g., U.S. Pat. No. 5,908,830; U.S. Patent Application 2007/0134735; U.S. Patent Application 2009/0215780, which are incorporated herein by reference.

Examples of medicaments that can be used to modulate heat loss in the neonatal mammalian subject and to modulate the thermogenic activity of brown adipocytes and reduce heat loss in the neonatal subject include exogenous thyroid-active substances that include, but are not limited to, T4 hormone and its analogs (L-thyroxine, levothyroxine, 3,5,3′,5′-tetraiodo-L-thyronine), T3 hormone and its analogs (3,5,3′-triiodo-L-thyronine, liothyronine, tertroxin, cytomel), T3/T4 combinations (liotrix, Thyrolar™), thyroid hormone precursors (thyroglobulin, proloid), thyroprotein, thyroactive iodinated casein, thyroid hormone, dessicated thyroid extracts (thyroidine). Substances that stimulate the thyroid to produce T4 or T3 can be administered, that include, but are not limited to, TSH (thyroid-stimulating hormone, thyrotropin, thyrotropic hormone, Ambinon™ or Dermathycin™), and TRH (thyrotropin-releasing hormone). See, e.g., U.S. Patent Application 2006/0008512; U.S. Patent Application 2002/0160394, which are incorporated herein by reference.

Medicaments can further include growth factors or other agents to promote the viability, proliferation, expansion, and/or differentiation of the transplanted brown adipocytes, or to promote vascularization and/or neuralization of the tissue comprising the transplanted brown adipocytes or precursors thereof. Examples of factors that positively affect adipose growth or differentiation include, but are not limited to, glucocorticoid, growth hormone, IGF-1, insulin, prostaglandins, or thyroid hormone, and other examples provided herein. Examples of angiogenic or arteriogenic growth factors that promote or stabilize vascularization include, but are not limited to, VEGF, FGF, angiopoietins, matrix metalloproteinase, Delta-like ligand 4, PDGF, TGF-β, plasminogen activators, or eNOS. See, e.g., Patrick, Anat. Rec. 263: 361-366, 2001, which is incorporated herein by reference. Examples of factors that promote neural outgrowth or stabilization include, but are not limited to, NGF, erythropoietin, as well as a number of the factors listed above.

Under conditions in which the transplanted brown adipocytes are from an allogeneic or xenogeneic origin, the medicaments can include one or more immunosuppressive agents to reduce and/or prevent rejection of the transplanted brown adipocytes. An immunosuppressive agent can be one or more of an agent of drug which inhibits or interferes with normal immune function. Examples of immunosuppressive agents that inhibit T-cell and/or B-cell costimulation pathways include, but are not limited to, cyclosporine A, tacrolimus, mycophenolate mofetil, rapamycin, or anti-thymocyte antibodies.

In general, medicaments can be administered on any of a number of dosing schedules depending upon the nature of the medicament. The medicament can be administered simultaneously with the injected brown adipocytes, e.g., in the same syringe, by separate local administration at the injection site, or by systemic administration. The medicament can be administered before, during and/or after injection of the brown adipocytes. The duration of treatment can be, e.g., approximately 1-6 days, approximately 1-4 weeks, or approximately 1-6 months. In an aspect, administration of the medicament can be discontinued once the neonatal subject achieves normal levels of thermoregulation. In the case of an immunosuppressive agent, the medicament may need to be administered on a regular basis over the lifetime of the transplanted cells which in some instances may be until the neonatal mammalian subject can thermoregulate normally without assistance from the transplanted brown adipocytes.

### PROPHETIC EXAMPLES

### Example 1

### Modulating Heat Loss In A Neonatal Human Subject By Administering Allogeneic Mature Brown Adipose Tissue

A method is described for modulating heat loss in a neonatal human subject. The method includes transplanting allogeneic mature brown adipose tissue provided by a related donor to a subcutaneous site in the recipient neonatal subject. Mature brown adipose tissue is harvested from the related donor, who is the mother of the neonatal human subject. The mature brown adipose tissue is transplanted to a subcutaneous site, e.g., the interscapular region, of the neonatal subject.

Mature brown adipose tissue is harvested from the mother of the neonatal human subject using liposuction. Depots of brown adipose tissue are located in the supraclavicular region of the mother using positron-emission tomography (PET) and computed tomography (CT) at ambient temperature of 17° C. in the presence of 18F-fluorodeoxyglucose. See, e.g., Virtanen, et al., N. Engl. J. Med. 360: 1518-1525, 2009, which is incorporated herein by reference. A local anesthetic, e.g., lidocaine, is used to anesthetize the site of harvest. An aspiration cannula connected to a syringe is inserted through the skin into or near the identified brown adipose tissue depot in the supraclavicular region of the mother of the neonatal human subject. The brown adipose tissue is suctioned by hand or with a mechanical vacuum. Suction is applied slowly to avoid excessive negative pressure, which can contribute to breakage and/or vaporization of the cells. A moderate amount of adipose tissue is harvested (e.g., from about 0.1 milliliters to about 50 milliliters of adipose tissue).

Once the aspirate of mature brown adipose tissue has been harvested, it can be directly injected into the neonate or further processed to remove maternal blood and other non-adipocyte components. The aspirate is washed in sterile normal saline (0.91% w/v of NaCl, ˜300 mOsm/L) by gently transferring the cells/tissue through multiple syringes using tulip connections to 1 mL tuberculin syringes. The adipocytes are resuspended in sterile normal saline in preparation for transplantation.

The resuspended brown adipocytes (approximately 1-2 million cells per ml) are loaded into a syringe fitted with an 18 gauge needle. Prior to subcutaneous injection of the brown adipocyte cell suspension at a site in the interscapular region of the neonatal subject, the site is anesthetized with a local anesthetic, for example, lidocaine. A single subcutaneous injection or multiple subcutaneous injections at varied sites in the interscapular region are performed, depending upon the volume and number of cells to be transplanted.

Body temperature measurements are used to monitor response to the transplanted brown adipose tissue in the neonatal human subject. The body temperature of the neonatal subject is monitored on a regular basis, e.g., every hour, using an axillary underarm thermometer (e.g., from Exergen Corporation, West

Yorkshire, UK). Temperatures in the range of 36.5° C. to 37.5° C. are considered appropriate for preterm neonates. Secondary measurements include monitoring for signs of hypothermia including skin temperature and pallor, respiration and heart rate, feeding behavior, and activity level. If the body temperature of the neonatal subject is not attaining a thermoregulated state, additional brown adipocytes may be administered to the internal site to increase thermoregulation in the neonatal subject. If the body temperature of the neonatal subject is not reaching thermoregulation, a possible cause may be lack of viability or rejection of the tissue. The viability of the transplanted cells is then monitored by positron-emission tomography (PET) and computed tomography (CT) in the presence of ¹⁸F-fluorodeoxyglucose as described by Virtanen, et al., (in N. Engl. J Med. 360:1518-1525, 2009, which is incorporated herein by reference). An immune response and/or rejection of the transplanted allogeneic cells can be monitored by assessing antibodies and other immune-related molecules against the donor cells. To avoid a donor-mediated immune response or graft versus host disease (GVHD), or to protect the allogeneic cells from an immune response, the brown adipocytes may be encapsulated within a non-immunogenic material. The brown adipocytes may be encapsulated in a combination of alginate and poly-L-lysine. See, e.g., Goren et al., FASEB Journal, 24: 22-31, 2010 and Machluf, et al., Endocrinology 144: 4975-4979, 2003, which are incorporated herein by reference. The degree of vascularization of the transplanted brown adipose can be monitored using Doppler ultrasound, either with or without a microbubble contrast agent. See, e.g., Krix, et al., Cancer Res. 63: 8264-8270, 2003, which is incorporated herein by reference.

### Example 2

### Modulating Heat Loss In A Neonatal Human Subject By Administering Allogeneic Pre-Adipocytes Cultured In Vitro In the Presence of Differentiation Factors

A method is described for modulating heat loss in a neonatal human subject. The method includes transplanting brown adipocytes to a subcutaneous site in the neonatal subject. The brown adipocytes are derived from in vitro differentiation of pre-adipocytes isolated from the mother of the neonatal subject and differentiated into brown adipocytes. Optimally, the isolation and differentiation of the mother's pre-adipocytes into brown adipocytes is performed prior to the birth of the neonatal human subject such that the cells are available for transplantation immediately upon delivery of the neonate. The brown adipocytes are encapsulated in alginate to provide immunoisolation of the cells and to prevent rejection of the cells by the neonatal subject. The encapsulated brown adipocytes derived from differentiation of pre-adipocytes of the mother are administered by subcutaneous injection into the interscapular region of the neonatal subject.

Pre-adipocytes for use in transplantation are isolated from subcutaneous white adipose tissue (WAT) of the mother of the neonatal subject. WAT from the mother is harvested using liposuction as described above. The adipose tissue is digested with collagenase (300 U/ml in phosphate buffered saline, 2% bovine serum albumin, pH 7.4) for 45 minutes under constant shaking. The mature white adipocytes are removed and the stromal vascular fraction is centrifuged, treated with erythrocyte lysis buffer (155 mM NH₄Cl, 5.7 mM K₂HPO₄, 0.1 mM EDTA, pH 7.3) and passed through 100, 70, and 40 μm sieves. Immunoselection/depletion is used to isolate CD34⁺/CD31⁻ cells, defined as pre-adipocyte progenitor cells. See, e.g., Elabd, et al., Stem Cells 27: 2753-2760, 2009, which is incorporated herein by reference.

The pre-adipocytes are cultured in RegES xeno-free medium supplemented with FGF to encourage proliferation and are grown to confluency. See, e.g., Rajala, et al., PLoS One. 5 (4): e10246, 2010, which is incorporated herein by reference. The expanded cell culture of pre-adipocytes is then transferred to pro-adipogenic culture medium to induce differentiation into brown adipocytes. The pro-adopogenic medium includes a combination of 10 μg/ml transferrin, 0.85 μM insulin, 0.2 nM triiodothyronine, 1 μM dexamethasone, 500 μM isobutyl methylxanthine (IBMX) and, optionally, 20-500 nM rosiglitazone. See, e.g., Elabd, et al., Stem Cells 27: 2753-2760, 2009, which is incorporated herein by reference. Over the course of 3 to 16 days, the differentiating progenitor cells are monitored for the expression of uncoupling protein-1 (UCP-1), a marker of brown adipocytes. UCP-1 expression is monitored by Western blot analysis with anti-UCP-1 specific antibodies (e.g., from Sigma-Aldrich, St. Louis, Mo.) or by a polymerase chain reaction (PCR) assay. A second marker of brown adipocytes is carnitine palmitoyltransferase (CPT1B). The CPT1B marker may be monitored by Western blot analysis or by polymerase chain reaction (PCR). The cells are also monitored for the emergence of lipid droplets within the cytoplasm as determined using Oil Red-O staining and light microscopy.

The differentiated brown adipocytes are further processed by encapsulation in alginate to provide immunoisolation of the cells from the immune system of the neonatal subject. The cells are encapsulated in alginate essentially as described by Tsai, et al., Biomed. Eng. Appl. Basis Comm. 18: 62-66, 2006, which is incorporated herein by reference. The differentiated brown adipocytes (10⁵cells/ml) are mixed with sodium alginate (e.g., from Sigma-Aldrich, St. Louis, Mo.) in phosphate buffered saline (PBS) and extruded from a syringe through a 23 gauge cannula into a 1.5% CaCl₂ solution to form spherical gel beads. The gel beads are collected and washed, and further coated with an aqueous solution of 0.1% poly(L-lysine) to stabilize the gel beads. The gel beads are incubated in culture medium containing transferrin, insulin, triiodothyronine, dexamethasone, and isobutyl methylxanthine as described above until transplantation into the neonatal subject.

The encapsulated differentiated brown adipocytes are injected into the interscapular area of the neonatal subject. The encapsulated brown adipocytes (approximately 1-2 million cells per ml) are washed and suspended in buffered saline, then loaded into a syringe fitted with an 18 gauge needle. Prior to subcutaneous injection of the brown adipocyte cell suspension at a site in the interscapular region of the neonatal subject, the site is anesthetized with a local anesthetic, for example, lidocaine. A single subcutaneous injection or multiple subcutaneous injections at varied sites in the interscapular region are done, depending upon the volume and number of cells to be transplanted. The viability and activity of the encapsulated brown adipocytes are monitored at one to two week intervals using gamma-camera imaging with ^{99m}Tc-tetrofosmin as described by Fukuchi, et al., J. Nucl. Med., 44: 1582-1585, 2003 which is incorporated herein by reference. For this analysis, ^{99m}Tc-tetrofosmin is injected into a peripheral vein of the neonatal subject and 45 minutes later scans are taken using the gamma-camera. In certain patients, including those at risk for cardiac problems, the metabolic activity of brown adipose tissue may also or instead be measured by the uptake of ¹⁸F-fluorodeoxyglucose (¹⁸F-FDG) and PET imaging. See, e.g., Lee, et al., Am J Physiol Endocrinol Metab 299: E601-E606, 2010; Saito, et al., Diabetes 58:1526-1531, 2009; Yoneshiro et al., Obesity (2011) 19: 13-16, 2011; van Marken Lichtenbelt, et al., N. Engl. J. Med., 360: 1500-1508, 2009; and Virtanen, et al., N. Engl. J. Med. 360: 1518-1525, 2009, which are each incorporated herein by reference.

Body temperature measurements are used to monitor response to the encapsulated differentiated brown adipocytes. The body temperature of the neonatal subject is monitored on a regular basis, e.g., every hour, using an axillary underarm thermometer (e.g., from Exergen Corporation, West Yorkshire, UK). Temperatures in the range of 36.5° C. to 37.5° C. are considered appropriate for preterm neonates. Secondary measurements include monitoring for signs of hypothermia including skin temperature and pallor, respiration and heart rate, feeding behavior, and activity level. Additional tests will be considered to assess viability and/or rejection of the transplants adipocytes as described herein.

### Example 3

### Modulating Heat Loss In A Neonatal Human Subject By Administering In Vitro Cultured Umbilical Cord Blood Stem Cells

A method is described for modulating heat loss in a neonatal human subject. The method includes transplanting autologous brown adipocytes derived from in vitro differentiation of mesenchymal stem cells isolated from tissue associated with the neonatal human subject. Mesenchymal stem cells are isolated from the cord blood of the neonatal subject immediately following delivery of the neonate. The mesenchymal stem cells are differentiated into brown adipocytes.

Umbilical cord blood is isolated from the neonatal subject during delivery of the neonate, then processed to obtain differentiated brown adipocytes. See, e.g., Kang, et al., Cell Biol. Int. 30: 569-575, 2006, which is incorporated herein by reference. The cord blood is diluted with an equal volume of buffer (containing phosphate buffered saline (PBS), pH 7.2, and 2 mM EDTA), and fractionated on a Ficoll-Paque™ (ρ=1.077 g/ml) density gradient medium by centrifugation at 800 g for 20 min. Mononuclear cells are recovered from the gradient interface and cultured in a xeno-free medium supplemented with GMP-certified human serum. See, e.g., Harmann et al., J Immunol Methods, 363 (1): 80-89 2010, which is incorporated herein by reference. Upon reaching 70% confluence, the mononuclear cells are treated with differentiation medium containing dexamethasone; insulin, isobutyl methylxanthine, and indomethacin. In some protocols for inducing differentiation, following growth to confluence, 20 nM to 100 nM rosiglitazone is substituted for dexamethasone and isobutyl methylxanthine in the growth medium. The mononuclear cells differentiate to brown adipocytes. See, e.g., Elabd, et al., Stem Cells 27: 2753-2760, 2009, which is incorporated herein by reference.

The differentiated brown adipocytes are injected into the interscapular region of the neonatal subject. The brown adipocytes (approximately 1-2 million cells per ml) are washed and resuspended in buffered saline, then loaded into a syringe fitted with an 18 gauge needle. Prior to subcutaneous injection of the brown adipocyte suspension at a site in the interscapular region of the neonatal subject, the site is anesthetized with a local anesthetic, for example, lidocaine. A single subcutaneous injection or multiple subcutaneous injections at varied sites in the interscapular region are done, depending upon the volume and number of cells to be transplanted.

Body temperature measurements are used to monitor response to the encapsulated differentiated brown adipocytes. The body temperature of the neonatal subject is monitored on a regular basis, e.g., every hour, using an axillary underarm thermometer (e.g., from Exergen Corporation, West Yorkshire, UK). Temperatures in the range of 36.5° C. to 37.5° C. are considered appropriate for preterm neonates.

Secondary measurements include monitoring for, signs of hypothermia including skin temperature and pallor, respiration and heart rate, feeding behavior, and activity level. Additional tests will be considered to assess viability and/or rejection of the transplanted adipocytes as described herein.

### Example 4

### Modulating Heat Loss In A Neonatal Human Subject By Administering Allogeneic Mature Brown Adipose Tissue In Combination With Medicament

A method is described for modulating heat loss in a neonatal human subject. The method includes transplanting allogeneic mature brown adipose tissue provided by a donor. The allogeneic mature brown adipose tissue is transplanted to a subcutaneous site in the neonatal subject. Mature brown adipose tissue is harvested from the mother of the neonatal human subject, and transplanted to a subcutaneous site at the nape of the neck of the neonatal subject. A medicament to control thermoregulation is administered in combination with the transplanted brown adipose tissue. The medicament administered for controlling thermoregulation is levothyroxine, a synthetic thyroid hormone. Thyroid hormone normally surges following full-term delivery and is necessary for the onset of independent thermoregulation. Rapid stimulation of thyroid hormone secretion contributes in part to increased expression of uncoupling protein 1 (UCP-1). In contrast, thyroid hormone levels in preterm infants younger than 30 weeks' gestation are considerably lower than full term infants. The postnatal surge of thyroid hormone levels normally observed in the first 24-48 hours is not observed in the preterm infants. In addition, the lower the hormone levels, the worse the prognosis for the neonate as measured by death or ventilator dependence at two weeks of age. See, e.g., Biswas, et al., Pediatrics, 109: 222-227, 2002; and Gate, et al., Experimental Physiology 85: 439-444, 2000, which are incorporated herein by reference.

Mature brown adipose tissue is harvested from the mother of the neonatal human subject by liposuction as described herein. Depots of brown adipose tissue are located in the supraclavicular region of the mother using positron-emission tomography (PET) and computed tomography (CT) at an ambient temperature of 17° C. in the presence of ¹⁸F-fluorodeoxyglucose as described by Virtanen, et al., N. Engl. J. Med. 360: 1518-1525, 2009, which is incorporated herein by reference. The mature brown adipocytes are prepared for transplantation as described herein.

The brown adipocytes isolated from mature brown adipose tissue are prepared in a manner so as to prevent an immune response by the immune system of the neonatal subject. The brown adipocytes are encapsulated in a combination of alginate and poly-L-lysine. See, e.g., Goren et al., FASEB Journal vol. 24 no. 1 22-31, 2010, and Machluf, et al., Endocrinology 144:4975-4979, 2003, which are incorporated herein by reference. The isolated mature brown adipocytes are suspended in a solution of sodium alginate and saline (1.2% weight/volume) to a final ratio of approximately 1-3×10⁶ cells per 1 ml of alginate. The suspension is sprayed through a 22-gauge needle into a calcium chloride solution. The resulting cell-calcium-alginate beads are washed several times in buffered saline and then collated with a 0.1% solution of poly-L-lysine. An additional coating of alginate can also be added.

The encapsulated brown adipocytes are injected into the interscapular region of the neonatal subject. The encapsulated brown adipocytes (approximately 1-2 million cells per ml) are washed and resuspended in buffered saline, then loaded into a syringe fitted with an 18 gauge needle. Prior to subcutaneous injection of the brown adipocyte cell suspension at a site in the interscapular region of the neonatal subject, the site is anesthetized with a local anesthetic, for example, lidocaine. A single subcutaneous injection or multiple subcutaneous injections at varied sites in the interscapular region are done, depending upon the volume and number of cells to be transplanted.

A medicament to control thermoregulation is administered in combination with the transplanted brown adipose tissue. The medicament for controlling thermoregulation is levothyroxine, a synthetic thyroid hormone, which is administered in conjunction,with transplantation of the mature brown adipose tissue. Levothyroxine sodium is administered to the neonatal subject as recommended in the prescribing information available from U.S. Food & Drug Administration (FDA). For neonates unable to swallow tablets, the tablet of levothyroxine sodium is finely crushed and resuspended in about 5 to 10 milliliters of water. The suspension is administered to the neonatal subject by dropper. The recommended starting dose of levothyroxine sodium for neonates is 10-15 micrograms/kilogram/day. Lower doses of levothryoxine can be considered depending upon the level of endogenous thyroid hormone.

Each recited range includes all combinations and sub-combinations of ranges, as well as specific numerals contained therein.

All publications and patent applications cited in this specification are herein incorporated by reference to the extent not inconsistent with the description herein and for all purposes as if each individual publication or patent application were specifically and individually indicated to be incorporated by reference for all purposes.

Those having ordinary skill in the art will recognize that the state of the art has progressed to the point where there is little distinction left between hardware and software implementations of aspects of systems; the use of hardware or software is generally (but not always, in that in certain contexts the choice between hardware and software can become significant) a design choice representing cost vs. efficiency tradeoffs. Those having ordinary skill in the art will recognize that there are various vehicles by which processes and/or systems and/or other technologies disclosed herein can be effected (e.g., hardware, software, and/or firmware), and that the preferred vehicle will vary with the context in which the processes and/or systems and/or other technologies are deployed. For example, if a surgeon determines that speed and accuracy are paramount, the surgeon may opt for a mainly hardware and/or firmware vehicle; alternatively, if flexibility is paramount, the implementer may opt for a mainly software implementation; or, yet again alternatively, the implementer may opt for some combination of hardware, software, and/or firmware. Hence, there are several possible vehicles by which the processes and/or devices and/or other technologies disclosed herein may be effected, none of which is inherently superior to the other in that any vehicle to be utilized is a choice dependent upon the context in which the vehicle will be deployed and the specific concerns (e.g., speed, flexibility, or predictability) of the implementer, any of which may vary. Those having ordinary skill in the art will recognize that optical aspects of implementations will typically employ optically-oriented hardware, software, and or firmware.

In a general sense the various aspects disclosed herein which can be implemented, individually and/or collectively, by a wide range of hardware, software, firmware, or any combination thereof can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices disclosed herein, or a microprocessor configured by a computer program which at least partially carries out processes and/or devices disclosed herein), electrical circuitry forming a memory device (e.g., forms of random access memory), and/or electrical circuitry forming a communications device (e.g., a modem, communications switch, or optical-electrical equipment). The subject matter disclosed herein may be implemented in an analog or digital fashion or some combination thereof.

The herein described components (e.g., steps), devices, and objects and the description accompanying them are used as examples for the sake of conceptual clarity and that various configuration modifications using the disclosure provided herein are within the skill of those in the art. Consequently, as used herein, the specific examples set forth and the accompanying description are intended to be representative of their more general classes. In general, use of any specific example herein is also intended to be representative of its class, and the non-inclusion of such specific components (e.g., steps), devices, and objects herein should not be taken as indicating that limitation is desired.

With respect to the use of substantially any plural or singular terms herein, the reader can translate from the plural to the singular or from the singular to the plural as is appropriate to the context or application. The various singular/plural permutations are not expressly set forth herein for sake of clarity.

The herein described subject matter sometimes illustrates different components contained within, or connected with, different other components. It is to be understood that such depicted architectures are merely examples, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality, and any two components capable of being so associated can also be viewed as being “operably couplable,” to each other to achieve the desired functionality. Specific examples of operably couplable include but are not limited to physically mateable or physically interacting components or wirelessly interactable or wirelessly interacting components or logically interacting or logically interactable components.

While particular aspects of the present subject matter described herein have been shown and described, changes and modifications may be made without departing from the subject matter described herein and its broader aspects and, therefore, the appended claims are to encompass within their scope all such changes and modifications as are within the true spirit and scope of the subject matter described herein. Furthermore, it is to be understood that the invention is defined by the appended claims. It will be understood that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an”; the same holds true for the use of definite articles used to introduce claim recitations. In addition, even if a specific number of an introduced claim recitation is explicitly recited, such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, Aand C together, B and C together, or A, B, and C together, etc.). Virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A method for modulating heat loss in a neonatal mammalian subject comprising:
providing exogenous brown adipocytes, or precursors thereof, to an internal site in the neonatal mammalian subject.

2. The method of claim 1, wherein the neonatal mammalian subject is a human, equine, bovine, ovine, swine, rodent, lagomorph, canine, or feline.

3. The method of claim 1, wherein the neonatal mammalian subject is a neonatal mammalian subject born preterm.

4. The method of claim 1, wherein providing the brown adipocytes comprises providing pre-adipocytes to the neonatal mammalian subject.

5. The method of claim 1, wherein the brown adipocytes, or the precursors thereof, are substantially purified.

6. The method of claim 1, comprising providing factors to the subject that promote at least one of adipocyte proliferation, adipocyte differentiation, or adipose angiogenesis.

7. The method of claim 6, wherein the factors include differentiation factors, growth factors, or angiogenic factors.

8. The method of claim 1, wherein modulating heat loss includes reducing heat loss in the neonatal mammalian subject by providing the exogenous brown adipocytes, or the precursors thereof, to the internal site in the neonatal mammalian subject.

9. The method of claim 1, wherein the precursors include one or more of stem cells, totipotent stem cells, multipotent stem cells, pluripotent stem cells, oligopotent stem cells, embryonic stem cells, de-differentiated stem cells, trans-differentiated stem cells, mesenchymal stem cells, adipose-derived stem cells, adipocyte progenitor cells, pre-adipocytes, myoblasts, muscle-derived stem cells, or bone marrow-derived stem cells.

10. The method of claim 1, wherein the exogenous brown adipocytes include mature brown adipocytes.

11. The method of claim 1, wherein the internal site includes one or more of a subcutaneous site, scapular site, axillary site, thoracic site, abdominal site, or blood vessel site of the neonatal mammalian subject.

12. The method of claim 1, wherein the brown adipocytes or the precursors thereof derive from one or more of cell donors, tissue donors, tissue culture stock, cell lines, or genetically manipulated cells.

13. The method of claim 12, wherein the genetically manipulated cells include an exogenous DNA sequence encoding one or more of a mammalian UCP polypeptide or a PRDM16 polypeptide.

14. The method of claim 12, wherein the one or more cell donors or tissue donors include a genetically related donor of the neonatal mammalian subject.

15. The method of claim 14, wherein the genetically related donor is a mother, father, sibling, grandparent, aunt, or uncle of the neonatal mammalian subject.

16. The method of claim 1, wherein the exogenous brown adipocytes or the precursors thereof derive from one or more of an autologous tissue, allogeneic tissue, or xenogeneic tissue.

17. The method of claim 1, wherein the exogenous brown adipocytes or the precursors thereof derive from a neonatal-associated tissue.

18. The method of claim 1, wherein the exogenous brown adipocytes or the precursors thereof derive from one or more of adipocytes, pre-adipocytes, stem cells, cord blood cells, placental cells, myoblasts, or bone marrow cells.

19. The method of claim 1, comprising expanding, maturing, or differentiating the exogenous brown adipocytes or the precursors thereof in vitro.

20. The method of claim 19, comprising providing one of more of differentiation factors or growth factors in vitro to the exogenous brown adipocytes or the precursors thereof.

21. The method of claim 1, wherein the brown adipocytes, or the precursors thereof include one or more detectable markers incorporated with the brown adipocytes or the precursors thereof.

22. The method of claim 1, wherein providing the brown adipocytes or the precursors thereof to the internal site comprises injecting the brown adipocytes or the precursors thereof.

23. The method of claim 22, comprising injecting the brown adipocytes or the precursors thereof in a pharmaceutically acceptable carrier.

24. The method of claim 1, wherein providing the brown adipocytes or the precursors thereof to the internal site comprises implanting the brown adipocytes or the precursors thereof.

25. The method of claim 1, comprising providing the brown adipocytes or the precursors thereof in a pharmaceutically acceptable carrier.

26. The method of claim 1, comprising providing the brown adipocytes or the precursors thereof in one or more biocompatible carriers.

27. The method of claim 26, comprising encapsulating the brown adipocytes or the precursors thereof.

28. The method of claim 26, comprising providing the brown adipocytes or the precursors thereof in an immunoisolating material.

29. The method of claim 26, wherein the biocompatible carrier includes at least one of a membrane, natural matrix, synthetic matrix, polymer, scaffold, hydrogel, natural sponge, synthetic sponge, microbead, microcapsule, microsphere, microparticle, or an encapsulating material.

30. The method of claim 1, comprising providing one or more medicaments for modulating heat loss from the brown adipocytes.

31. The method of claim 30, wherein the one or more medicaments include one or more of a neurotransmitter, a neurotrophic agent, a neuropeptide, an adipokine, or an uncoupling protein.

32. The method of claim 31, wherein the one or more medicaments include one or more of a β3-adrenergic receptor agonist, NPY antagonist, leptin, UCP activating agent, thyroxine, serotonin reuptake inhibitor, MCH antagonist, GLP-1 agonist, 5-HT_{2C} agonist, 5-HT_{2A} agonist, galanin antagonist, CRF agonist, urocortin agonist, melanocortin agonist or enterostatin agonist.

33. A method comprising:
providing exogenous brown adipocytes, or precursors thereof, to an internal site in a neonatal mammalian subject.

34. (canceled)

35. The method of claim 33, wherein the neonatal mammalian subject is a neonatal mammalian subject born preterm.

36. The method of claim 33, wherein providing the brown adipocytes comprises providing pre-adipocytes to the neonatal mammalian subject.

37. (canceled)

38. The method of claim 36, comprising providing factors to the subject that promote at least one of adipocyte proliferation, adipocyte differentiation, or adipose angiogenesis.

39. The method of claim 38, wherein the factors include differentiation factors, growth factors, or angiogenic factors.

40. The method of claim 33, wherein modulating heat loss includes reducing heat loss in the neonatal mammalian subject by providing the exogenous brown adipocytes, or the precursors thereof, to the internal site in the neonatal mammalian subject.

41. (canceled)

42. The method of claim 33, wherein the exogenous brown adipocytes include mature brown adipocytes.

43. (canceled)

44. The method of claim 33, wherein the brown adipocytes or the precursors thereof derive from one or more of cell donors, tissue donors, tissue culture stock, cell lines, or genetically manipulated cells.

45. The method of claim 44, wherein the genetically manipulated cells include an exogenous DNA sequence encoding one or more of a mammalian. UCP polypeptide or a PRDM16 polypeptide.

46. The method of claim 44, wherein the one or more cell donors or tissue donors include a genetically related donor of the neonatal mammalian subject.

47. (canceled)

48. The method of claim 33, wherein the exogenous brown adipocytes or the precursors thereof derive from one or more of an autologous tissue, allogeneic tissue, or xenogeneic tissue.

49. The method of claim 33, wherein the exogenous brown adipocytes or the precursors thereof derive from a neonatal-associated tissue.

50. The method of claim 33, wherein the exogenous brown adipocytes or the precursors thereof derive from one or more of adipocytes, pre-adipocytes, stem cells, cord blood cells, placental cells, myoblasts, or bone marrow cells.

51. The method of claim 33, comprising expanding, maturing or differentiating the exogenous brown adipocytes or the precursors thereof in vitro.

52. The method of claim 51, comprising providing one or more of differentiation factors or growth factors in vitro to the exogenous, brown adipocytes or the precursors thereof.

53. The method of claim 33, wherein the brown adipocytes or the precursors thereof include one or more detectable markers incorporated with the brown adipocytes or the precursors thereof.

54. The method of claim 33, wherein providing the brown adipocytes or the precursors thereof to the internal site comprises injecting the brown adipocytes or the precursors thereof.

55. The method of claim 33, comprising injecting the brown adipocytes or the precursors thereof in a pharmaceutically acceptable carrier.

56. The method of claim 33, wherein providing the brown adipocytes or the precursors thereof to the internal site comprises implanting the brown adipocytes or the precursors thereof.

57. (canceled)

58. The method of claim 33, comprising providing the brown adipocytes or the one or more precursors thereof in one or more biocompatible carriers.

59. The method of claim 58, comprising encapsulating the brown adipocytes or the precursors thereof.

60. The method of claim 58, comprising providing the brown adipocytes or the one or more precursors thereof in an immunoisolating carrier.

61.-64. (canceled)
